(19) United States
(12) Patent Application Publication
(10) Pub. No.: US 2016/0256508 A1
(43) Pub. Date: Sep. 8, 2016

(54) COMPOSITIONS CONTAINING NITRO FATTY ACIDS

(71) Applicant: Nitromega Corp., Pittsburgh, PA (US)

(72) Inventor: Raymond A. Miller, Magnolia, OH (US)

(21) Appl. No.: 15/157,674

(22) Filed: May 18, 2016

Related U.S. Application Data
(63) Continuation of application No. 14/129,332, filed on Dec. 26, 2013, now abandoned.
Application No. 14/129,332 is a continuation-in-part of application No. 13/174,206, filed on Jun. 30, 2011, now Pat. No. 8,937,194.
Application No. 13/174,206 is a continuation-in-part of application No. 12/651,079, filed on Dec. 31, 2009, now Pat. No. 8,933,255.
(60) Provisional application No. 61/503,461, filed on Jun. 30, 2011.
(60) Provisional application No. 61/506,978, filed on Jul. 12, 2011.
(60) Provisional application No. 61/558,813, filed on Nov. 11, 2011.
(60) Provisional application No. 61/141,844, filed on Dec. 31, 2008.

(51) Int. Cl. A61K 36/63; A61K 35/60; A61K 36/28; A61K 31/201

(52) U.S. Cl. CPC A61K 36/63; A61K 31/201; A61K 35/60; A61K 36/28; A23L 1/3008; A23L 1/3006; A23V 2002/00

(57) ABSTRACT

Compositions, dietary supplement compositions, and topical compositions including activated fatty acids and methods for using activated fatty acids to treat a variety of diseases.

### CROSS-REFERENCE TO RELATED APPLICATION

This application claims the benefit of U.S. Provisional Patent Application Ser. No. 61/503,461, filed on Jun. 30, 2011, U.S. Provisional Patent Application Ser. No. 61/506,978, filed on Jul. 12, 2011, U.S. Provisional Patent Application Ser. No. 61/558,813, filed on Nov. 11, 2011, and U.S. Continuation-In-Part patent application Ser. No. 13/174,206, filed Jun. 30, 2011.

### SUMMARY

Embodiments of the invention presented herein are directed to nutritional or dietary supplements, topical formulations, such as salves and lotions, and other nutraceutical compositions that include one or more activated fatty acids such as for example, nitro fatty acids.

In some embodiments, the nutraceutical supplements and topical formulations may include one or more nutraceutical other than nitro fatty acids such as rice bran oil, enzyme-treated stabilized rice bran, a solubilized fraction of rice bran oil, and derivatives thereof, glucosamine derivatives, methylsulfonylmethane, yucca concentrate, grape seed extract, beta-carotene, ephedra, ginko biloba, goldenseal, valerian, ginseng, and echinacea. The activated fatty acids may be isolated from a natural source such as fish oil and may be derived from omega-3 fatty acids, conjugated linoleic acid, linoleic acid, α-linoleic acid, oleic acid, eicosapentaenoic acid, docosahexaenoic acid or a derivative or combination thereof. In particular embodiment, the nutraceuticals may further include non-nitrated fatty acids.

For topical formulations the nutraceutical may include a dermatalogically acceptable vehicle, and in certain embodiments other nutraceuticals such as, for example, hyaluronic acid, chondroitin sulphate, collagen glucosamine, keratan sulphate, dermatan sulphate, vitamin C, green tea extract, shea butter, grape-seed extract, aloe extract, or mixtures thereof.

For dietary and nutritional supplement embodiments, the nutraceutical may be provided as a gel capsule, and in some embodiments, the nutritional supplement may be an additive for food.

Some embodiments of the invention are directed to the selection, formulation, and use of compounds which act with a protective response to prevent and attenuate inflammation to provide a therapeutic effect in their control of the pathological inflammation processes, and are also important in providing useful biochemical tools for mechanistic investigation of the enzymes involved.

Some embodiments of the invention are directed to the topical use of nitroalkene compositions, including particularly, conjugated nitro-linoleic acid, nitro-linoleic acid, nitro-oleic acid, nitrated species of arachidonic acid and nitrated cholesteryl lineoleate, as lipi signaling mediatos to reduce inflammation and inflammation mediated skin conditions.

Some embodiments of the invention provide therapeutically effective topical compositions of nitroalkene and carrier to prevent, treat, or otherwise improve the skin conditions through topical application.

Some embodiments of the invention provide methods for preventing and/or treating skin damage that comprise applying a composition containing nitroalkene in a dermatologically acceptable carrier to skin.

In accordance with the present invention, topical methods of use of nitroalkenes to prevent or treat rosacea, eczema, psoriasis, xerosis, dermatitis, seborrhea, acne, alopecia, other types of skin inflammation, skin aging, and scarring are disclosed.

The amount of nitroalkene necessary to treat skin or prevent skin damage is not fixed per se and is necessarily dependent upon the amount and identity of any adjunct ingredients in the preparation. In some typical embodiments of the invention, the composition comprises about 0.025% to about 70% by weight nitroalkene in a dermatologically acceptable polymer polyether and/or phosphatidycholine carrier. Optionally, at least one or a mixture of lipoic acid, fatty acid ester of ascorbic acid may be added to the composition.

In some typical embodiments of the invention, the method for preventing and/or treating skin damage comprises applying a composition containing about 0.025% to about 70% by weight of nitroalkene in a dermatologically acceptable carrier. Optionally, at least one or a mixture of lipoic acid or fatty acid ester of ascorbic acid may be added to the composition.

Some embodiments are directed to a dietary supplement including a fatty acid component enriched for one or more activated fatty acids fatty acids and a nutraceutically acceptable excipient. In some embodiments, the activated fatty acid may be derived from an omega-3 fatty acid, an omega-6 fatty acid, an omega-9 fatty acid, and combinations thereof. In other embodiments, the activated fatty acid may be a nitro-fatty acid, conjugated nitro-fatty acid, or a keto-fatty acid, and in particular embodiments, the activated fatty acid may be nitro-linoleic acid, nitro-α-linoleic acid, nitro-γ-linoleic acid, nitro-oleic acid, nitro-eicosapentaenoic acid, nitro-docosahexaenoic acid, conjugated nitro-linoleic acid, keto-linoleic acid, keto-linoleic acid, keto-γ-linoleic acid, keto-α-linoleic acid, keto-oleic acid, keto-eicosapentaenoic acid, keto-docosahexaenoic acid or a derivative or combination thereof. In still other embodiments, the dietary supplement may also include one or more of linoleic acid, α-linoleic acid, γ-linoleic acid, oleic acid, eicosapentaenoic acid (EPA), docosahexaenoic acid (DHA), conjugated linoleic acid, or derivatives thereof. In some embodiment, the dietary supplement may further include one or more nutraceutical selected from vitamin A, vitamin B, vitamin B-1, vitamin B-2, vitamin B-6, vitamin B-12, vitamin C, vitamin D, vitamin D3, vitamin E, selenium, β-carotene, ginko biloba, goldenseal, valerian, ginseng, echinacea, grape seed extracts, ephedra, yucca concentrates, green tea extract, rice bran extract, wheat germ, wheat germ extract, beeswax, red yeast rice extract, stevia leaf extract, flaxseed oil, borage seed oil, coenzyme Q10, glucosamine derivatives, methylsulfonylmethane, pantothenic acid, biotin, thiamin, riboflavin, niacin, folic acid, palmitic acid, and derivatives thereof.

In particular embodiments, the dietary supplement may include a first fatty acid component enriched for one or more activated fatty acid selected from nitro-linoleic acid, keto-linoleic acid, nitro-oleic acid, and keto-oleic acid and a second fatty acid component having one or more non-activated fatty acid selected from linoleic acid, γ-linoleic acid, α-linoleic acid, oleic acid, eicosapentaenoic acid (EPA), docosahexaenoic acid (DHA), conjugated linoleic acid, or derivatives thereof, and in some embodiments, the dietary supplement may further include vitamin E or a derivative thereof. In other embodiments, the dietary supplement may include one or more secondary agent including but not limited to vitamin A, vitamin B, vitamin B-1, vitamin B-2, vitamin B-6, vitamin B-12, vitamin C, vitamin D, vitamin D3, vitamin E, selenium, β-carotene, ginko biloba, goldenseal, valerian, ginseng, echinacea, grape seed extracts, ephedra, yucca concentrates, green tea extract, rice bran extract, wheat germ, wheat germ extract, beeswax, red yeast rice extract, stevia leaf extract, flaxseed oil, borage seed oil, coenzyme Q10, glucosamine derivatives, methylsulfonylmethane, pantothenic acid, biotin, thiamin, riboflavin, niacin, folic acid, palmitic acid, and derivatives thereof. In some embodiments, the dietary supplement may include one or more secondary agent selected from policosanols, guggulipds, rice bran extract, wheat germ, wheat germ extract, beeswax, and red yeast rice extract, and such a dietary supplement may be formulated to promote a healthy heart and circulatory system. In other embodiments, the dietary supplement may include one or more secondary agent selected from vitamin B-1, vitamin B-2, vitamin 8-6, vitamin B-12, vitamin C, vitamin D, vitamin D3, vitamin E, selenium, goldenseal, valerian, ginseng, and echinacea and such a dietary supplement may be formulated to promote healthy cell proliferation. In still other embodiments, the dietary supplement may include one or more secondary agent selected from vitamin A, vitamin C, vitamin E, and β-carotene, and such a dietary supplement may be formulated to promote healthy eyes. In yet other embodiments, the dietary supplement may include one or more secondary agent selected from vitamin A, vitamin C, vitamin E, selenium, ginko biloba, goldenseal, valerian, ginseng, echinacea, ephedra, green tea extract, and yucca concentrate, and such a dietary supplement may be formulated to promote general health.

Various embodiments of the invention are also directed to pharmaceutical composition. In such embodiments, the one or more nitro fatty acids may make up about 10% by weight to about 95% by weight. As above, the pharmaceutical compositions may include one or more nutraceutical other than nitro fatty acids such as, for example, rice bran oil, enzyme-treated stabilized rice bran, a solubilized fraction of rice bran oil, and derivatives thereof; glucosamine derivatives, methylsulfonylmethane, yucca concentrate, grape seed extract, beta-carotene, ephedra, ginko biloba, goldenseal, valerian, ginseng, and echinacea. The activated fatty acid may be derived from an omega-3 fatty acids, omega-6 fatty acids, omega-7 fatty acids, omega-9 fatty acids, α-linoleic acid, γ-linoleic acid, oleic acid, conjugated linoleic acid, linoleic acid, eicosapentaenoic acid, docosahexaenoic acid or a derivative or combination thereof, and may contain non-activated fatty acids. Such pharmaceutical compositions may be topical compositions, and in some embodiments, the compositions may further include other agents such as solubilizers, stabilizers, colorants, plasticizers diluents, fillers, disintegrants, binders, lubricants, surfactants, hydrophobic vehicles, water soluble vehicles, emulsifiers, buffers, humectants, moisturizers, antioxidants, preservatives or combinations thereof. In still other embodiments, the composition, may further include one or more secondary agents such as, for example, antioxidants, statins, squalene synthesis inhibitors, azetidinone-based compounds, LDL catabolism activators, PPAR antagonists or agonists, antiarrhythmic agent, NSAIDs and nutraceutical equivalents thereof.

Further embodiments are directed to methods for improving the health of an individual by administering to the individual a dietary supplement including a fatty acid component enriched for one or more activated fatty acids, and a nutraceutically acceptable excipient. In some embodiments, the dietary supplement may include a first fatty acid component enriched for one or more activated fatty acid selected from conjugated nitro-linoleic acid, nitro-linoleic acid, keto-linoleic acid, nitro-oleic acid, and keto-oleic acid and a second fatty acid component having one or more non-activated fatty acid selected from linoleic acid, α-linoleic acid, γ-linoleic acid, oleic acid, eicosapentaenoic acid (EPA), docosahexaenoic acid (DHA), conjugated linoleic acid, or derivatives thereof, and in particular embodiments, the dietary supplement may further include vitamin E or a derivative thereof. In some embodiments, the dietary supplement may further include one or more secondary agent selected from vitamin A, vitamin B, vitamin B-1, vitamin B-2, vitamin B-6, vitamin B-12, vitamin C, vitamin D, vitamin D3, vitamin E, selenium, β-carotene, ginko biloba, goldenseal, valerian, ginseng, echinacea, grape seed extracts, ephedra, yucca concentrates, green tea extract, rice bran extract, wheat germ, wheat germ extract, beeswax, red yeast rice extract, stevia leaf extract, flaxseed oil, borage seed oil, coenzyme Q10, glucosamine derivatives, methylsulfonylmethane, pantothenic acid, biotin, thiamin, riboflavin, niacin, folic acid, palmitic acid, and derivatives thereof. In some embodiments, the dietary supplement may include one or more secondary agent selected from policosanols, guggulipds, rice bran extract, wheat germ, wheat germ extract, beeswax, and red yeast rice extract, and such a dietary supplement may be formulated to promote a healthy heart and circulatory system. In other embodiments, the dietary supplement may include one or more secondary agent selected from vitamin B-1, vitamin B-2, vitamin B-6, vitamin B-12, vitamin C, vitamin D, vitamin D3, vitamin E, selenium, goldenseal, valerian, ginseng, and echinacea and such a dietary supplement may be formulated to promote healthy cell proliferation. In still other embodiments, the dietary supplement may include one or more secondary agent selected from vitamin A, vitamin C, vitamin E, and β-carotene, and such a dietary supplement may be formulated to promote healthy eyes. In yet other embodiments, the dietary supplement may include one or more secondary agent selected from vitamin A, vitamin C, vitamin E, selenium, ginko biloba, goldenseal, valerian, ginseng, echinacea, ephedra, green tea extract, and yucca concentrate, and such a dietary supplement may be formulated to promote general health.

Embodiments of the invention also include methods for preparing a nitro-fatty acid by isolating nitro fatty acids from fish oil. Other methods for preparing a nitro fatty acid include the steps of contacting an existing unsaturated fatty acid with a nitro containing compound; and reacting an existing unsaturated fatty acid with a nitro containing compound to form a nitro fatty acid. Still other methods for preparing activated fatty acids include the steps of contacting an unsaturated fatty acid with a mercuric salt and a selenium compound; contacting an intermediate resulting from step 1 with an electron withdrawing group donating reagent; reacting the intermediate resulting from step 2 with an oxidizing agent. Yet other methods for preparing nitro fatty acids include the steps of combining a first component at least comprising an aliphatic hydrocarbon having an electron withdrawing group at one end and a second component at least comprising aliphatic hydrocarbon chain having an aldehyde at one end in the presence of a base to form a first intermediate; generating an alkene from the first intermediate.

### DESCRIPTION OF DRAWINGS

FIG. 1 is a graph showing stability of 10-nitro oleic acid in olive oil over a period of 19 days at 22° C., 37° C. and 50° C. Stability is plotted as a percentage of the starting concentration of 10-nitro oleic acid.

### BACKGROUND

Nitric oxide (NO) is an endogenously generated, lipophilic signaling molecule that has been implicated in the maintenance of vascular homeostasis, modulation of oxygen radical reactions, inflammatory cell function, post-translational protein modification and regulation of gene expression. In addition, nitric oxide-derived species display separate and unique pharmacological properties, specifically can mediate oxidation and nitration of biomolecules such as, for example, unsaturated fatty acids.

Various reactions yield products capable of concerted oxidation, nitrosation and nitration of target molecules. For example, nitric oxide may react with superoxide (O₂⁻) to yield peroxynitrite (ONOO⁻) and its conjugate acid, peroxynitritrous acid (ONOOH), the latter of which may undergo homolytic scission to form nitrogen dioxide (.NO₂) and hydroxyl radical (.OH). In some instances, biological conditions may favor the reaction of ONOO⁻ with CO₂ which yields nitrosoperoxycarbonate (ONOOCO₂⁻), which rapidly yields .NO₂ and carbonate (.CO₃⁻) radicals via homolysis or rearrangement to NO₃⁻ and CO₂. During inflammation, neutrophil myeloperoxidase and heme proteins such as myoglobin and cytochrome c catalyze H₂O₂-dependent oxidation of nitrite (NO₂⁻) to NO₂ resulting in biomolecule oxidation and nitration that is influenced by the spatial distribution of catalytic heme proteins. The reaction of .NO with O₂ can also produce products that can be substrates or reactants for nitrosation and nitration. For example, the small molecular radius, uncharged nature and lipophilicity of .NO and O₂ facilitate concentration of these species in biological membranes in a process referred to as the “molecular lens” effect. The increase in concentration induced by .NO and O₂ solvation in hydrophobic cell compartments accelerates the normally slow reaction of .NO with O₂ to yield N₂O₃ and N₂O₄. Finally, environmental sources also yield .NO₂ as a product of photochemical air pollution and tobacco smoke.

Nitration of fatty acids by .NO₂ can occur through several methods. For example, during both basal cell signaling and tissue inflammatory conditions, .NO₂ can react with membrane and lipoprotein lipids. In both in vivo and in vitro systems, .NO₂ has been shown to initiate radical chain auto-oxidation of polyunsaturated fatty acids via hydrogen abstraction from the bis-allylic carbon to form nitrous acid and a resonance-stabilized bis-allylic radical. Depending on the radical environment, the lipid radical species can react with molecular oxygen to form a peroxyl radical, which can react further to form lipid hydroperoxides then oxidized lipids. During inflammation or ischemia, when O₂ levels are lower, lipid radicals can react to an even greater extent with .NO₂ to generate multiple nitration products including singly nitrated, nitrohydroxy- and dinitro-fatty acid adducts. These products can be generated via hydrogen abstraction, direct addition of .NO₂ across the double bond, or both, and in some cases, such reactions may be followed by further reactions of the intermediate products that are formed. Hydrogen abstraction causes a rearrangement of the double bonds to form a conjugated diene; however, the addition of .NO₂ maintains a methylene-interrupted diene configuration to yield singly nitrated polyunsaturated fatty acids. This arrangement is similar to nitration products generated by the nitronium ion (NO₂⁺), which can be produced by ONOO⁻ reaction with heme proteins or via secondary products of CO₂ reaction with ONOO⁻.

The reaction of polyunsaturated fatty acids with acidified nitrite (HNO₂) can generate a complex mixture of products similar to those formed by direct reaction with .NO₂, including the formation of singly nitrated products that maintain the bis-allylic bond arrangement. The acidification of NO₂⁻ can create a labile species, HNO₂, which is in equilibrium with secondary products, including N₂O₃, .NO and .NO₂, all of which can participate in nitration reactions. The relevance of this pathway as a mechanism of fatty acid nitration is exemplified by physiological and pathological conditions wherein NO₂⁻ is exposed to low pH (e.g., <pH 4.0). This may conceivably occur in the gastric compartment, following endosomal or phagolysosomal acidification or in tissues following-post ischemic reperfusion.

Nitrated linoleic acid (LNO₂) and conjugated nitro-linoleic acid (CLNO₂) have been shown to display robust cell signaling activities that are generally anti-inflammatory in nature. Synthetic LNO₂ can inhibit human platelet function via cAMP-dependent mechanisms and inhibits neutrophil O₂⁻ generation, calcium influx, elastase release, CD11b expression and degranulation via non-cAMP, non-cGMP-dependent mechanisms. LNO₂ may also induce vessel relaxation in part via cGMP-dependent mechanisms. In aggregate, these data, derived from a synthetic fatty acid infer that nitro derivatives of fatty acids (NO₂-FA) represent a novel class of lipid-derived signaling mediators. To date, a gap in the clinical detection and structural characterization of nitrated fatty acids has limited defining NO₂-FA derivatives as biologically-relevant lipid signaling mediators that converge .NO and oxygenated lipid signaling pathways.

The metabolism of arachidonic acid is a key element of inflammation. In acute inflammation, there is typically a respiratory burst of neutrophil activity that initiates cascades involving a change in the oxidation state of the cell. Alteration in the redox state of the cell activates transcription factors such as NFκB as well as API, which then causes production of proinflammatory mediators. These mediators, such as Tumor necrosis factorA (TFα) and various interleukins, cause a burst of other cytokines. Arachadonic acid is released, which is oxidized to biologically active mediators. When arachadonic acid is oxidized via the cyclooxygenase or lipoxygenase pathways, eicosanoids e.g. prostaglandins, leukotrines, and hyroxyeicosatetraenoic acid (HETE) are produced, which cause erythma, edema, and free radical production.

Acute inflammation is often characterized by the generation of excited oxygen species, e.g. superoxide anion, which damages the lipid-rich membranes and activate the chemical mediators of the proinflammation and inflammation cascades. These oxygenated species tend to concentrate in hydrophobic regions. Both in or near these hydrophobic compartments, .NO and NOx undergo a rich spectrum of reactions with oxygen species, transition metals, thiols, lipids, and a variety of organic radicals. These multifaceted reactions yield reactive species that transduce .NO signaling and modulate tissue inflammatory responses.

During inflammation, adaptive and protective responses are elicited by vascular and other tissues to protect the host from its own mechanisms directed at destroying invading pathogens. Heme oxygenase 1 (HO-1) plays a central role in vascular inflammatory signaling and mediates a protective response to inflammatory stresses such as atherosclerosis, acute renal failure, vascular restenosis, transplant rejection, and sepsis. Heme oxygenase 1 catalyzes the degradation of heme to billverdin, iron, and CO, the last of which has been shown to display diverse, adaptive biological properties, including anti-inflammatory, anti-apoptotic, and vasodilatory actions. During inflammation, HO-1 gene expression is up-regulated, with induction typically occurring transcriptionally. Neutrophil myeloperoxidase and heme proteins such as myoglobin and cytochrome c catalyze H₂O₂-dependent oxidation of nitrite (NO₂) to NO₂, resulting in biomolecule oxidation and nitration that is influenced by the spatial distribution of catalytic heme proteins. These and other products are capable of concerted oxidation, nitrosation and nitration of target molecules.

The body contains an endogenous antioxidant defense system made up of antioxidants such as vitamins C and E, glutathione, and enzymes, e.g., superoxide dismutase. When metabolism increases or the body is subjected to other stress such as infection, extreme exercise, radiation (ionizing and non-ionizing), or chemicals, the endogenous antioxidant systems are overwhelmed, and free radical damage takes place. Over the years, the cell membrane continually receives damage from reactive oxygen species and other free radicals, resulting in cross-linkage or cleavage or proteins and lipoproteins, and oxidation of membrane lipids and lipoproteins. Damage to the cell membrane can result in myriad changes including loss of cell permeability, increased intercellular ionic concentration, and decreased cellular capacity to excrete or detoxify waste products. As the intercellular ionic concentration of potassium increases, colloid density increases and m-RNA and protein synthesis are hampered, resulting in decreased cellular repair. Some cells become so dehydrated they cannot function at all.

Natural products such as Royal Jelly comprises water, crude protein, including small amounts of many different amino acids, simple sugars (monosaccharides), and fatty acids. It also contains many trace minerals, some enzymes, antibacterial and antibiotic components and vitamins. The major type of fatty acids contained in Royal Jelly are hydroxyl fatty acids such as 10-hydroxy-2-decenoic acid. Royal Jelly is harvested from bees and has been reported as a possible immunomodulatory agent in Graves' disease. It has also been reported to stimulate the growth of glial cells and neural stem cells in the brain. To date, there is preliminary evidence that it may have some cholesterol-lowering, anti-inflammatory, wound-healing, and antibiotic effects. Research also suggests that the 10-Hydroxy-2-decenoic acid may inhibit the vascularization of tumors and has also been hypothesized to correct cholesterol level imbalances due to nicotine consumption. Holistically, Royal Jelly is believed to have anti-aging properties making it a component in skin care and natural beauty products.

It would be desirable to have topical treatments for rosacea, eczema, acne, alopecia, psoriasis and inflammatory conditions in general using compositions which disrupt the inflammatory cascades describes above.

### DETAILED DESCRIPTION

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meaning as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference in their entirety. Nothing herein is to be construed as an admission that the invention is not entitled to antedate such disclosure by virtue of prior invention.

It must also be noted that as used herein and in the appended claims, the singular forms “a”, “an”, and “the” include plural reference unless the context clearly dictates otherwise. Thus, for example, reference to a “cell” is a reference to one or more cells and equivalents thereof known to those skilled in the art, and so forth.

As used herein, the term “about” means plus or minus 0.10% of the numerical value of the number with which it is being used. Therefore, about 50% means in the range of 45%-55%.

“Administering” when used in conjunction with a therapeutic means to administer a therapeutic directly into or onto a target tissue or to administer a therapeutic to a patient, whereby the therapeutic positively impacts the tissue to which it is targeted. Thus, as used herein, the term “administering”, when used in conjunction with a nitrated lipid can include, but is not limited to, providing a nitrated lipid to a subject systemically by, for example, intravenous injection, whereby the therapeutic reaches the target tissue. “Administering” a composition may be accomplished by, for example, injection, oral administration, topical administration, or by these methods in combination with other known techniques. Such combination techniques include heating, radiation, ultrasound and the use of delivery agents.

The term “animal” as used herein includes, but is not limited to, humans and non-human vertebrates such as wild, domestic and farm animals.

The term “improves” is used to convey that the present invention changes either the characteristics and/or the physical attributes of the tissue to which it is being provided, applied or administered. The term “improves” may also be used in conjunction with a diseased state such that when a diseased state is “improved” the symptoms or physical characteristics associated with the diseased state are diminished, reduced or eliminated.

The term “inhibiting” includes the administration of a compound of the present invention to prevent the onset of the symptoms, alleviating the symptoms, or eliminating the disease, condition or disorder.

By “pharmaceutically acceptable”, it is meant the carrier, diluent or excipient must be compatible with the other ingredients of the formulation and not deleterious to the recipient thereof.

“Nutraceutical” as used herein generally refer to natural, bioactive chemical compounds that provide physiological benefits, including, disease prevention and health promotion which may be used to supplement the diet. Nutraceuticals can be either purified or concentrated by using bioengineering methods and can be enhanced through genetic methods, which contain elevated levels of natural substances. Examples of nutraceuticals include isolated nutrients and herbal products and generally contain at least one of the following ingredients: a vitamin, a mineral, an herb or other botanical, an amino acid, a metabolite, constituent, extract, or combination of these ingredients. Common examples of nutraceuticals include beta-carotene, ephedra, ginko biloba, goldenseal, valerian, ginseng, and echinacea. The nutraceuticals described herein may be useful for maintenance and support of, for example, healthy joints, skin, and eye and brain function.

As used herein, the term “therapeutic” means an agent utilized to treat, combat, ameliorate, prevent or improve an unwanted condition or disease of a patient. In part, embodiments of the present invention are directed to the treatment of inflammation, obesity-related diseases, metabolic diseases, cardiovascular diseases, cerebrovascular and neurodegenerative diseases, cancer or the aberrant proliferation of cells.

A “therapeutically effective amount” or “effective amount” of a composition is a predetermined amount calculated to achieve the desired effect, i.e., to inhibit, block, or reverse the activation, migration, or proliferation of cells. The activity contemplated by the present methods includes both medical therapeutic and/or prophylactic treatment, as appropriate. The specific dose of a compound administered according to this invention to obtain therapeutic and/or prophylactic effects will, of course, be determined by the particular circumstances surrounding the case, including, for example, the compound administered, the route of administration, and the condition being treated. However, it will be understood that the effective amount administered will be determined by the physician in the light of the relevant circumstances including the condition to be treated, the choice of compound to be administered, and the chosen route of administration, and therefore, the above dosage ranges are not intended to limit the scope of the invention in any way. A therapeutically effective amount of compound of this invention is typically an amount such that when it is administered in a physiologically tolerable excipient composition, it is sufficient to achieve an effective systemic concentration or local concentration in the tissue.

The terms “treat,” “treated,” or “treating” as used herein refers to both therapeutic treatment and prophylactic or preventative measures, wherein the object is to prevent or slow down (lessen) an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

As used herein and in the attached claims, the term “enriched” shall mean that the composition or portion of the composition includes a concentration of the identified component that is greater than the amount of the component naturally occurring in the composition. For example, with reference to activated fatty acids a composition enriched for activated fatty acids may include greater than at least 50 nM activated fatty acids. Therefore, a composition that is enriched for activated fatty acids may be at least 0.05% by weight activated fatty acid, at least 0.1% by weight activated fatty acid, at least 0.15% by weight activated fatty acid, at least 0.25% by weight activated fatty acid, at least 0.5% by weight activated fatty acid, at least 1.0% by weight activated fatty acid, at least 2% by weight activated fatty acid, and so on.

Generally speaking, the term “tissue” refers to any aggregation of similarly specialized cells which are united in the performance of a particular function.

Embodiments of the invention presented herein are generally directed to activated fatty acids and, in particular, activated unsaturated fatty acids. As used herein an “activated fatty acid” refers to a fatty acid having at least one electron withdrawing group covalently bound to a carbon of the saturated or unsaturated aliphatic chain of a fatty acid. Such activated fatty acids may be substituted by any number of electron withdrawing groups at any number of positions on the hydrocarbon chain, and an electron withdrawing group may be positioned in either cis or trans configuration at a double bond or in either R or S absolute stereochemistry at an sp³ chiral/stereogenic center. For example, in one embodiment, an activated fatty acid may have one electron withdrawing group, and in another, an activated fatty acid may be substituted with multiple electron withdrawing groups at multiple positions along the hydrocarbon chain. While the activated fatty acids of the invention may have an electron withdrawing group positioned at any carbon along the aliphatic hydrocarbon chain between the carboxy terminal carbon to the terminal methyl (ω), in some embodiments, the electron withdrawing group may be positioned within about 1 carbon from the carboxy terminal carbon and within about 1 carbon from the terminal methyl. In other embodiments, the electron withdrawing group may be positioned within about 3 carbons of either the carboxy terminal carbon and/or the methyl terminal carbon, and in still others embodiments, the electron withdrawing group may be positioned within 5 carbons of either of the carboxy terminal carbon and/or the methyl terminal carbon.

In certain embodiments, the electron withdrawing group may be positioned on a carbon directly attached to a double bond of the activated fatty acid forming an “electron withdrawing vinyl” group. The electron withdrawing group of such vinyl groups may be on either side of the double bond. Fatty acids encompassed by embodiments of the invention may have one or more than one electron withdrawing vinyl groups at any carbon on the aliphatic hydrocarbon chain, and there are several ways that an unsaturated fatty acid can have one electron-withdrawing group. In one embodiment, an activated oleic acid (ocatadecac-9-enoic acid) which is an 18 carbon, ω-6 fatty acid with one double bond (denoted “18:1”) between the 6^{th} (C-13) and 7^{th} (C-12) carbons, may have an electron withdrawing group at either C-13 or C-12. In another exemplary embodiment, an activated linoleic acid (octadeac-9,12,-dienoic acid), which is an 18 carbon, ω-6 fatty acid with two double bonds (denoted “18:2”) between the 6th (C-13) and 7^{th} (C-12) carbons and the 9^{th} (C-10) and 10^{th} (C-9) carbons, may have an electron withdrawing group at C-9 or C-10 or C-12 or C-13. Similarly, other polyunsaturated fatty acids, with 3, 4, 5, 6 or more double bonds, can have one electron withdrawing at either position on any of the double bond carbons, including all possible permutations of positions and electron-withdrawing groups.

In other embodiments, a mono or polyunsaturated fatty acid may have two electron-withdrawing groups, and there are several ways that an unsaturated fatty acid can have two electron-withdrawing groups. For example, in one embodiment, an activated oleic acid (ocatadecac-9-enoic acid) which is an 18 carbon, ω-6 fatty acid with one double bond (denoted “18:1”) between the 6^{th} (C-13) and 7^{th} (C-12) carbons, may have an electron withdrawing group at both C-13 and C-12. In another exemplary embodiment, an activated linoleic acid (octadeac-9,12,-dienoic acid), which is an 18 carbon, ω-6 fatty acid with two double bonds (denoted “18:2”) between the 6^{th} (C-13) and 7^{th} (C-12) carbons and the 9^{th} (C-10) and 10^{th} (C-9) carbons, may have an electron withdrawing group at any two of the positions C-9, C-10, C-12 or C-13, with the following possible permutations: C-9 and C-10, C-9 and C-12, C-9 and C-13, C-10 and C-12, C-10 and C-13, or C-12 and C-13. In yet another exemplary embodiment, an activated conjugated linoleic acid ((9Z,11E)-octadeca-9,11-dienoic acid), which is an 18 carbon, ω-7 fatty acid with two double bonds (denoted “18:2”) between the 7^{th} (C-12) and 8^{th} (C-11) carbons and the 9^{th} (C-10) and 10^{th} (C-9) carbons, may have an electron withdrawing group at any two of the positions C-9, C-10, C-11 or C-12, with the following possible permutations: C-9 and C-10, C-9 and C-12, C-9 and C-11, C-10 and C-12, C-10 and C-11, or C-12 and C-11. Similarly, other polyunsaturated fatty acids, with 3, 4, 5, 6 or more double bonds, can have two electron withdrawing at two of the positions on any of the double bond carbons, including all possible permutations of positions and electron-withdrawing groups.

In analogy to the preceding descriptions of compounds with one electron-withdrawing group or two electron-withdrawing groups, it is also possible to have three, four, five or more electron withdrawing groups. Following the same logic above, in the preceding descriptions of compounds with one electron-withdrawing group or two electron-withdrawing groups, polyunsaturated fatty acids, with 3, 4, 5, 6 or more double bonds, can have multiple electron withdrawing (three, four, five or more, as available positions for substitution permit) at any of the positions on any of the double bond carbons, including all possible permutations of positions and electron-withdrawing groups.

The term “electron-withdrawing group” is recognized in the art and denotes the tendency of a substituent to attract valence electrons from neighboring atoms, i.e., the substituent is electronegative with respect to neighboring atoms. A quantification of the level of electron-withdrawing capability is given by the Hammett sigma (σ) constant (see, e.g., J. March, Advanced Organic Chemistry, McGraw Hill Book Company, New York, (1977 edition) pp. 251-259). The Hammett constant values are generally negative for electron donating groups and positive for electron withdrawing groups. For example the Hammet constant for para substituted NH₂ (σ [P]) is about −0.7 and the σ [P] for a nitro group is about 0.8.

Embodiments of the invention encompass any known electron withdrawing group. For example, electron-withdrawing groups may include, but are not limited to, aldehyde (—COH) acyl (—COR), carbonyl (—CO), carboxylic acid (—COOH), ester (—COOR), halides (—Cl, —F, —Br, etc.), fluoromethyl (—CFₙ), cyano (—CN), sulfonyl (—SOₙ), sulfone (—SO₂R), sulfonic acid (−SO₃H), 1°, 2° and 3° ammonium (−NR₃+), and nitro (—NO₂). In some embodiments, the electron withdrawing group may be a strong electron withdrawing group having a σ of at least about 0.2, and in certain embodiments, the electron withdrawing group may form a dipole. For example, in particular embodiments, the electron withdrawing group may be a nitro, ammonium or sulfonyl. In other embodiments, the activated fatty acids of the invention may be additionally substituted by non-electron withdrawing groups or electron donating groups including, for example, alcohol (—OH), reverse ester (—OOCR), alkyl, alkenyl, alkynyl, 1° and 2° amines (—NR₂), nitrate (—ONO₂), nitrito (—ONO) and the like.

The fatty acids of various embodiments may be any unsaturated and polyunsaturated fatty acid known in the art. The term “fatty acid” describes aliphatic monocarboxylic acids. Various embodiments include nitrated fatty acid having an aliphatic hydrocarbon chain identical or similar to identified, naturally occurring fatty acids. For example, aliphatic hydrocarbon chains of known naturally occurring fatty acids are generally unbranched and contain an even number of from about 4 to about 24 carbons. Embodiments of the invention encompass such naturally occurring fatty acids as well as non-naturally occurring fatty acids which may contain an odd number of carbons and/or a non-naturally occurring linker. Some embodiments of the invention include fatty acids having from 8 to 23 carbons, and others include fatty acids having from 12 to 18 carbons in the aliphatic hydrocarbon chain. In still other embodiments, fatty acids may have greater than 24 carbons in the aliphatic hydrocarbon chain. The fatty acids of the invention may also be branched at one or more location along the hydrocarbon chain, and in various embodiments, each branch may include an aliphatic hydrocarbon chain of from 1 to 24 carbons, 2 to 20 carbons or 4 to 18 carbons.

The aliphatic hydrocarbon chain of fatty acids of various embodiments may be unsaturated or polyunsaturated. The term “unsaturated” refers to a fatty acid having a aliphatic hydrocarbon chain that includes at least one double bond and/or substituent. In contrast, a “saturated” hydrocarbon chain does not include any double bonds or substituents. Thus, each carbon of the hydrocarbon chain is ‘saturated’ and has the maximum number of hydrogens. “Polyunsaturated,” generally, refers to fatty acids having hydrocarbon chains with more than one double bond. The double bonds of the unsaturated or polyunsaturated fatty acids of various embodiments may be at any location along the aliphatic hydrocarbon chain and may be in either cis or trans configuration. The term “cis,” refers to a double bond in which carbons adjacent to the double bond are on the same side and the term “trans” refers to a double bond in which carbons adjacent to the double bond are on opposite sides. Typically “cis” is the same as Z, and “trans” is the same as E but sometimes the UPAC rules for naming compounds will give the opposite of this, which is the typical case in nitroalkenes. For example, a nitroalkene can have the two carbon groups “cis” but the two groups that take priority for the naming of compounds (a nitro group on one carbon of the alkene and a carbon group on the other carbon of the alkene) are on opposite sides and thus are E. Therefore the nitroalkene analog of a “cis” double bond is actually an E nitroalkene. Similarly, the nitroalkene analog of a “trans” double bond is actually a Z nitroalkene. Without wishing to be bound by theory, double bonds in cis configuration along the carbon chain (cis carbon chain but E nitroalkene) may induce a bend in the hydrocarbon chain. Double bonds in “trans,” configuration along the carbon chain (trans carbon chain but Z nitroalkene) may not cause the hydrocarbon chain to bend.

Many unsaturated and polyunsaturated fatty acids have been identified and are known to be naturally occurring. Such unsaturated or polyunsaturated naturally occurring fatty acids, generally, include an even number of carbons in their aliphatic hydrocarbon chain. For example, a naturally occurring unsaturated or polyunsaturated fatty acid may have, 4, 6, 8, 10, 12, 14, 16, 18, 20, 22 and so on carbons and may include omega (ω-3, ω-5, ω-6, ω-7, ω-9 fatty acids and the like. Any such fatty acid may be useful in embodiments of the invention. The symbol ‘ω’ is used to refer to the terminal methyl carbon of the aliphatic hydrocarbon chain. The placement of the double bond of the ω-X fatty acid is the carbon-carbon bond X number of carbons from the co carbon. For example, an ω-6 fatty acid has a double bond between the 6th and 7^{th} carbons counting backward from the co carbon and an ω-3 fatty acid has a double bond between the 3^{rd} and 4^{th} carbons counting backward from the co carbon. Various embodiments of the invention include nitrated ω-3 fatty acids, including, but not limited to, linolenic acid, alpha-linolenic acid, eicosapentanoic acid, docosapentaenoic acid, docosahexanoic acid and stearidonic acid; nitrated ω-5 fatty acids including, but not limited to, myristoleic acid; nitrated ω-6 fatty acids including, but not limited to, linoleic acid, gamma-linoleic acid, dihomo-gamma-linoleic acid and arachidonic acid; nitrated ω-7 fatty acids including, but not limited to, conjugated linoleic acid, palmitoleic acid; and nitrated ω-9 fatty acids including, but not limited to, oleic acid and erucic acid. Of course, the fatty acids of the invention may also be referred to using IUPAC nomenclature in which the placement of the double bond is determined by counting from the carbon of the carboxylic acid, and ‘C—X’ denotes the carbon in aliphatic hydrocarbons using IUPAC nomenclature wherein X is the number of the carbon counting from the carboxylic acid. Embodiments of the invention also include synthetic equivalents to naturally occurring fatty acids and derivatives thereof.

In particular embodiments, the fatty acids utilized in embodiments of the invention may be omega-3 fatty acids. As used herein, the term “omega-3 fatty acids” or “0-3 fatty acids” may include natural or synthetic omega-3 fatty acids, or pharmaceutically acceptable esters, derivatives, conjugates (see, e.g., U.S. Publication No. 2004/0254357 to Zaloga et al. and U.S. Pat. No. 6,245,811 to Horrobin et al., each of which is hereby incorporated by reference in its entirety), precursors or salts thereof and mixtures thereof. Examples of ω-3 fatty acid oils include but are not limited to ω-3 polyunsaturated, long-chain fatty acids such as a eicosapentaenoic acid (EPA), docosahexaenoic acid (DHA), and α-linolenic acid; esters of ω-3 fatty acids with glycerol such as mono-, di- and triglycerides; and esters of the ω-3 fatty acids and a primary, secondary or tertiary alcohol such as fatty acid methyl esters and fatty acid ethyl esters. In certain embodiments, the ω-3 fatty acid oils may be long-chain fatty acids such as EPA or DHA, triglycerides thereof, ethyl esters thereof and mixtures thereof. The ω-3 fatty acids or their esters, derivatives, conjugates, precursors, salts and mixtures thereof can be used either in their pure form or as a component of an oil, such as fish oil, preferably purified fish oil concentrates.

Various fish oils are known and useful as sources for ω-3, ω-6, and ω-9 fatty acids, and any such oil may be used in embodiments of the invention. For example, oils derived from herring, sardines, mackerel, lake trout, flounder, albacore tuna, krill, and salmon are useful sources of ω-3, ω-6, and ω-9 fatty acids.

Commercially available ω-3 fatty acids suitable for use in the invention may include, but are not limited to, Life's DHA (Martek Biosciences Corporation. Columbia, Md.) Incromega F2250, F2628, E2251, F2573, TG2162, TG2779, TG2928, TG3525 and E5015 (Croda International PLC, Yorkshire, England), and EPAX6000FA, EPAX5000TG, EPAX4510TG, EPAX2050TG, K85TG, K85EE, K80EE and EPAX7010EE (Pronova Biocare a.s., 1327 Lysaker, Norway). In certain embodiments, the ω-3 fatty acids may be a mixture of several ω-3 fatty acids such as OMACOR™ omega-3 fatty acids which are combinations of EPA and DHA ω-3 fatty acids, and are described in U.S. Pat. Nos. 5,502,077, 5,656,667 and 5,698,594, which are hereby incorporated by reference in their entireties.

Similarly various plant oils are known and useful as sources for ω-3, ω-6, and ω-9 fatty acids, and any such oil may be used in embodiments of the invention. For example, olive oil, peanut oil, grape seed oil, sea buckthorn oil, sesame oil, and f poppyseed oil are useful sources of ω-3, ω-6, and ω-9 fatty acids, and in particular ω-9 fatty acids, such as, oleic acid. http://en.wikipedia.org/wiki/Oleic_acid-cite_note-pmid17093176-2#cite_note-pmid17093176-2

In particular embodiments of the invention the fatty acids utilized in embodiments of the invention may be conjugated fatty acids. As used herein, the term “conjugated fatty acid” may include natural or synthetic conjugated fatty acids, or pharmaceutically acceptable esters, derivatives, conjugates, precursors or salts thereof and mixtures thereof. As the nomenclature implies, the double bonds of CLAs are conjugated, with only one single bond between them. Examples of conjugated fatty acids include (9Z,11E)-octadeca-9,11-dienoic acid and 10E, 12Z-octadeca-10,12-dienoic acid, both of which are isomers of conjugated linoleic acid an ω)-7 polyunsaturated fatty acid.

Conjugated fatty acids such as conjugated linoleic acid can be readily found in nature. In particular, in meat and dairy products derived from ruminant animals. Conjugated linoleic acid is also produced by humans from certain trans isoforms of oleic acid.

In another embodiment of the invention, activated forms of conjugated fatty acids may be used such as, for example (9Z,11E)-9- and 12-nitro-octadeca-9,11-dienoic acid and 10E, 12Z-10 and 13-nitro-octadeca-10,12-dienoic acid. Nitroalkene derivatives of linoleic acid (nitrolinoleic acid) are formed via nitric oxide-dependent oxidative inflammatory reactions and are found at concentrations of ≈500 nM in the blood of healthy individuals.

Other embodiments of the invention include unsaturated or polyunsaturated non-naturally occurring fatty acids which may have an odd number of carbons such as, for example, 5, 7, 9, 11, 13, 15, 17, 19, 20, 21 and so on. As in naturally occurring fatty acids, the one or more double bonds associated with non-naturally occurring fatty acids may be at any position along the aliphatic hydrocarbon chain, and the double bonds may be in either cis or trans configuration. In yet other embodiments, the non-naturally occurring fatty acids may include one or more linker groups which interrupt the aliphatic hydrocarbon chain. For example, in some embodiments, activated fatty acids may have one or more non-carbon-carbon linkage such as, for example, ester, ether, vinyl ether, amino, imine and the like at any position within the alipatic hydrocarbon chain.

For example, embodiments of the invention include compounds of general formulae I and II:

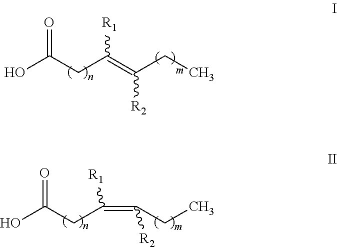

wherein R₁ and R₂ are independently selected from —H and any electron withdrawing groups including, but not limited to —OH, —COH, —COR, —CO, —COOH, —COOR, —Cl, —F, —Br, —I, —CF₃, —CN, —SO₃⁻, —SO₂R, —SO₃H, —NH₃⁺, —NH₂R⁺, —NHR₂⁺, —NR₃⁺ and —NO₂⁻ wherein at least one of R₁ and R₂ is an electron withdrawing group and m and n are, independently, 1-20. Some embodiments include compounds of general formula III;

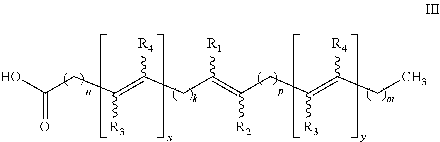

wherein R₁, R₂, m and n are as described above, R₃ and R₄ are, independently, selected from —H, —OH, —COH, —COR, —CO, —COOH, —COOR, —Cl, —F, —Br, —I, —CF₃, —CN, —SO₃⁻, —SO₂R, —SO₃H, —NH₃⁺, —NH₂R⁺, —NHR₂⁺, —NR₃⁺ and —NO₂⁻, k and p are, independently, 0 to 5 and x and y are independently, 0 to 3, and wherein each double bond is in either cis or trans configuration. In still other embodiments, any carbon associated with m, n, k or p may be substituted. Some embodiments include compounds of general formula IV:

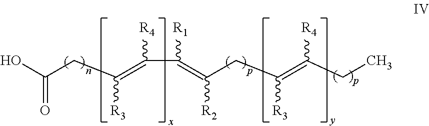

wherein R₁, R₂, m and n are as described above, R₃ and R₄ are, independently, selected from —H, —OH, —COH, —COR, —CO, —COOH, —COOR, —Cl, —F, —Br, —I, —CF₃, —CN, —SO₃⁻, —SO₂R, —SO₃H, —NH₃⁺, —NH₂R⁺, —NHR₂⁺, —NR₃⁺ and —NO₂⁺, k and p are, independently, 0 to 5 and x and y are independently, 0 to 3, and wherein each double bond is in either cis or trans configuration. In still other embodiments, any carbon associated with m, n, or p may be substituted.

The activated fatty acids described above may be prepared as a pharmaceutically acceptable formulation. The term “pharmaceutically acceptable” is used herein to mean that the compound is appropriate for use in a pharmaceutical product. For example, pharmaceutically acceptable cations include metallic ions and organic ions. More preferred metallic ions include, but are not limited to, appropriate alkali metal salts, alkaline earth metal salts and other physiological acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include, without limitation, hydrochloric acid, hydroiodic acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid, oxalacetic acid, fumaric acid, propionic acid, aspartic acid, glutamic acid, benzoic acid, and the like.

Isomeric and tautomeric forms of activated fatty acids of the invention as well as pharmaceutically acceptable salts of these compounds are also encompassed by the invention. Exemplary pharmaceutically acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, beta.-hydroxybutyric, galactaric and galacturonic acids.

Suitable pharmaceutically acceptable base addition salts used in connection with the activated fatty acids of the invention include metallic ion salts and organic ion salts. Exemplary metallic ion salts include, but are not limited to, appropriate alkali metal (group Ia) salts, alkaline earth metal (group IIa) salts and other physiological acceptable metal ions. Such salts can be made from the ions of aluminum, calcium, lithium, magnesium, potassium, sodium and zinc. Preferred organic salts can be made from tertiary amines and quaternary ammonium salts, including in part, trimethylamine, diethylamine, N,N′-dibenzylethylenediamine, chloroprocaine, choline, diethanolamine, ethylenediamine, meglumine (N-methylglucamine) and procaine. All of the above salts can be prepared by those skilled in the art by conventional means from the corresponding compound of the present invention.

Activated fatty acids as described in various embodiments of the invention above, may be administered to individuals to treat, ameliorate and/or prevent a number both acute and chronic inflammatory and metabolic conditions. In particular embodiments, activated fatty acids may be used to treat acute conditions including general inflammation, arterial stenosis, organ transplant rejection and burns, and chronic conditions such as, chronic lung injury and respiratory distress, diabetes, hypertension, obesity, rheumatoid arthritis, neurodegenerative disorders and various skin disorders. However, in other embodiments, activated fatty acids may be used to treat any condition having symptoms including chronic or acute inflammation, such as, for example, arthritis, lupus, Lyme's disease, gout, sepsis, hyperthermia, ulcers, enterocolitis, osteoporosis, viral or bacterial infections, cytomegalovirus, periodontal disease, glomerulonephritis, sarcoidosis, lung disease, lung inflammation, fibrosis of the lung, asthma, acquired respiratory distress syndrome, tobacco induced lung disease, granuloma formation, fibrosis of the liver, graft vs. host disease, postsurgical inflammation, coronary artery bypass graft (CABG), acute and chronic leukemia, B lymphocyte leukemia, neoplastic diseases, arteriosclerosis, atherosclerosis, myocardial inflammation, psoriasis, immunodeficiency, disseminated intravascular coagulation, systemic sclerosis, amyotrophic lateral sclerosis, multiple sclerosis, Parkinson's disease, Alzheimer's disease, encephalomyelitis, edema, inflammatory bowel disease, hyper IgE syndrome, cancer metastasis or growth, adoptive immune therapy, reperfusion syndrome, radiation burns, alopecia and the like.

For example, in one embodiment, an activated fatty acid may be administered to treat hypertension by lowering blood pressure to normal levels without reducing the blood pressure of the individual below normal levels even if the activated fatty acid is over-administered. Thus, without wishing to be bound by theory, the activated fatty acids of the invention may provide treatment of an individual without the negative effects associated with over-administration or over-treatment using traditional medications.

In a still further embodiment, activated fatty acids may be useful for ischemic preconditioning or protecting the heart from ischemic injury due to vessel spasm or blockage. For example, nitrated fatty acids produced by mitochondria in cells under ischemic conditions cause a number of physiological changes within the cell that increases cell survival under ischemic conditions. By providing activated fatty acids to an individual, similar ischemic preconditioning or protection may be achieved allowing for improved survival of, for example, cardiac tissue under ischemic conditions or organs being preserved for optimizing viability and function upon transplantation. In particular embodiments, nutraceuticals including activated fatty acids may be provided to individuals at risk of heart disease, heart attack, heart failure, vascular blockage, arrhythmia, atrial fibrillation, heart valve diseases, cardiomyopathy, and the like to both reduce or alleviate the symptoms of such maladies and to increase the likelihood of survival in the event of, for example, a heart attack, arrhythmia, or arterial fibrillation or to more generally improve heart or circulatory system function.

In addition, activated fatty acid administration may be useful for activating a number of other factors important for cell signaling. For example, in one embodiment, activated fatty acids may be administered to induce gene expression and tissue activity of heme oxygenase-1 (HO-1) which has been shown to mediate adaptive and protective responses during inflammation, and activation of an adaptive or protective inflammatory response mediated by HO may be useful in treating inflammatory diseases such as, but not limited to, atherosclerosis, acute renal failure, vascular restenosis, transplant rejection, and sepsis. Thus, activated fatty acids may be useful for treating general inflammation resulting from surgery, injury or infection.

The nutraceuticals of the invention can be administered in any conventional manner by any route where they are active. Administration can be systemic or local. For example, administration can be, but is not limited to, parenteral, subcutaneous, intravenous, intramuscular, intraperitoneal, transdermal, oral, buccal, ocular, intravaginally, or inhalation. In certain embodiments, the administration may be parenteral. In some embodiments, the nutraceutical may be prepared in the presence or absence of stabilizing additives that favors extended systemic uptake, tissue half-life and intracellular delivery. Thus, modes of administration for the compounds of the present invention (either alone or in combination with other pharmaceuticals) can be injectable (including short-acting, depot, implant and pellet forms injected subcutaneously or intramuscularly). In some embodiments, an injectable formulation including an activated fatty acid may be deposited to a site of injury or inflammation, such as, for example, the site of a surgical incision or a site of inflammation due to arthroscopy, angioplasty, stent placement, by-pass surgery and so on.

When administered, activated fatty acids may interact with a number of cellular receptors and/or proteins that mediate inflammation, either by inhibiting or stimulating their activity thereby inhibiting or reducing inflammation. Without wishing to be bound by theory, activated fatty acids may modulate important signaling activities including, for example, neurotransmission, gene expression, vascular function and inflammatory responses, and chemical properties of activated fatty acids that may facilitate these activities include, but are not limited to, the strong, reversible electrophilic nature of the 3 carbon adjacent to the electron withdrawing vinyl group, an ability to undergo Nef-like acid base reactions to release NO, an ability to partition into both hydrophobic and hydrophilic compartments, and a strong affinity for G-protein coupled receptors and nuclear receptors.

For example, in one embodiment, activated fatty acids may be administered to mediate cell signaling via multiple G-protein coupled receptors and nuclear receptors such as, but not limited to, peroxisome proliferator-activated receptors (PPAR) including PPARα, PPARγ, and PPARδ. PPAR is a nuclear receptor that is expressed throughout an organism, including in monocytes/macrophages, neutrophils, endothelial cells, adipocytes, epithelial cells, hepatocytes, mesangial cells, vascular smooth muscle cells, neuronal cells and when “activated” induces transcription of a number of target genes. Activation of PPAR has been shown to play various roles in regulating tissue homeostasis including, for example, increasing insulin sensitivity, suppress chronic inflammatory processes, reduce circulating free fatty acid levels, correct endothelial dysfunction, reduce fatty streak formation, delay plaque formation, limit blood vessel wall thickening and enhance plaque stabilization and regression. The activated fatty acids embodied herein may perform each of these functions associated with PPAR activation.

Moreover, activated fatty acids may perform these functions without significantly altering normal cellular process. For example, in one embodiment, an activated fatty acid may be administered to treat hypertension by lowering blood pressure to normal levels without reducing the blood pressure of the individual below normal levels even if the activated fatty acid is over-administered. Thus, without wishing to be bound by theory, the activated fatty acids of the invention may provide treatment of an individual without the negative effects associated with over-administration or over-treatment using traditional medications.

Shopfer et al. have found that conjugated linoleic is an endogenous ligand for PPARγ. Activation of PPAR has been shown to be induced by a locking reaction in which a critical thiol in a highly conserved cysteine (Cys 285 of human PPARγ) which is located in a ligand binding domain of PPAR. Partial activation of PPAR has been shown to occur when relatively high concentrations of known thiol reactive compounds, such as 15-deoxy-Δ^{12,14}-prostaglandin J₂ (15-d PGJ₂), are administered. Without wishing to be bound by theory, activated fatty acids may bind to PPAR covalently at the reactive thiol in the ligand binding domain of PPAR. Moreover, activated fatty acids may induce a conformational change in PPAR. More specifically, activated fatty acid binding may result in the C-terminus of the ligand binding domain (α-helix 12) to adopt an active conformation that may promote a beneficial pattern of ω-repressor release and ω-activator recruitment. Thus, activated fatty acids may enhance PPAR activation and transcription of PPAR regulated genes beyond that of known PPAR activating compounds.

Activation of PPAR has been shown to be induced by a locking reaction in which a critical thiol in a highly conserved cysteine (Cys 285 of human PPARγ) which is located in a ligand binding domain of PPAR. Partial activation of PPAR has been shown to occur when relatively high concentrations of known thiol reactive compounds, such as 15-deoxy-Δ^{12,14}-prostaglandin J₂ (15-d PGJ₂), are administered. Without wishing to be bound by theory, activated fatty acids may bind to PPAR covalently at the reactive thiol in the ligand binding domain of PPAR. Moreover, activated fatty acids may induce a conformational change in PPAR. More specifically, activated fatty acid binding may result in the C-terminus of the ligand binding domain (α-helix 12) to adopt an active conformation that may promote a beneficial pattern of ω-repressor release and ω-activator recruitment. Thus, activated fatty acids may enhance PPAR activation and transcription of PPAR regulated genes beyond that of known PPAR activating compounds.

In addition to activation of PPAR, activated fatty acid administration may be useful for activating a number of other factors important for cell signaling. For example, in one embodiment, activated fatty acids may be administered to induce gene expression and tissue activity of heme oxygenase-1 (HO-1) which has been shown to mediate adaptive and protective responses during inflammation, and activation of an adaptive or protective inflammatory response mediated by HO may be useful in treating inflammatory diseases such as, but not limited to, atherosclerosis, acute renal failure, vascular restenosis, transplant rejection, and sepsis. In addition, activated fatty acid administration may be useful for activating a number of other factors important for cell signaling. For example, in one embodiment, activated fatty acids may be administered to induce gene expression and tissue activity of heme oxygenase-1 (HO-1) which has been shown to mediate adaptive and protective responses during inflammation, and activation of an adaptive or protective inflammatory response mediated by HO may be useful in treating inflammatory diseases such as, but not limited to, atherosclerosis, acute renal failure, vascular restenosis, transplant rejection, and sepsis. Thus, activated fatty acids may be useful for treating general inflammation resulting from surgery, injury or infection. In another embodiment, activated fatty acids may induce a reversible post-translational modification of proteins, such as, for example, glutathione (GSH) and glyceraldehyde-3-phosphate dehydrogenase (GAPDH) by covalently binding to catalytic cysteines on such proteins. Without wishing to be bound by theory, the covalent modification of these proteins by activated fatty acids may increase the hydrophobicity of these proteins inducing translocation to membranes and suggests a role for redox regulation of enzyme function, cell signaling and protein trafficking. In yet another embodiment, activated fatty acids may be administered to repress NF-κB dependent gene expression and endothelial tumor necrosis factor-α induced expression of vascular cell adhesion molecules in monocytes and macrophages which results in inhibition of rolling and adhesion during inflammation. Thus, activated fatty acids may be useful for treating general inflammation resulting from surgery, injury or infection. In a further embodiment, activated fatty acids may be administered to limit tissue inflammatory injury and inhibit the proliferation of vascular smooth muscle cells by increasing cellular levels of nuclear factor erythroid 2-related factor-2 (Nrf-2) which may be useful in the treatment of a number of vascular diseases. In a still further embodiment, activated fatty acids may be useful for ischemic preconditioning. For example, nitrated fatty acids produced by mitochondria in cells under ischemic conditions cause a number of physiological changes within the cell that increases cell survival under ischemic conditions. By providing activated fatty acids to an individual, similar ischemic preconditioning may be achieved allowing for improved survival of, for example, cardiac tissue under ischemic conditions or organs being preserved for optimizing viability and function upon transplantation.

The composition of the embodiments of the invention can be administered in any conventional manner by any route where they are active. Administration can be systemic or local. For example, administration can be, but is not limited to, parenteral, subcutaneous, intravenous, intramuscular, intraperitoneal, transdermal, oral, buccal, or ocular routes, or intravaginally, by inhalation, by depot injections, or by implants. In certain embodiments, the administration may be parenteral or intravenous, all in the presence or absence of stabilizing additives that favor extended systemic uptake, tissue half-life and intracellular delivery. Thus, modes of administration for the compounds of the present invention (either alone or in combination with other pharmaceuticals) can be injectable (including short-acting, depot, implant and pellet forms injected subcutaneously or intramuscularly). In some embodiments, an injectable formulation including an activated fatty acid may be deposited to a site of injury or inflammation, such as, for example, the site of a surgical incision or a site of inflammation due to arthroscopy, angioplasty, stent placement, by-pass surgery and so on.

In certain other embodiments, the compounds of the invention may be applied locally as a salve or lotion applied directly to an area of inflammation. For example, in some embodiments, a lotion or salve including activated fatty acids of the invention may be prepared and applied to a burn, radiation burn, site of dermal disorder, edema, arthritic joint or the like. Such salves and lotions, may include a topical formulation of one or more activated fatty acid in a dermatologically acceptable vehicle, and in particular embodiments, the topical formulation may as a nutraceutical salve or lotion which may contain for example, hyaluronic acid, chondroitin sulphate, collagen glucosamine, keratan sulphate, dermatan sulphate, vitamin C, green tea extract, shea butter, grape-seed extract, aloe extract, or mixtures thereof.

Various embodiments, of the invention are also directed to method for administering activated fatty acids. Specific modes of administration may vary and may depend on the indication. The selection of the specific route of administration and the dose regimen may be adjusted or titrated by the clinician according to methods known to the clinician in order to obtain the optimal clinical response. The amount of compound to be administered is that amount which is therapeutically effective. The dosage to be administered will depend on the characteristics of the subject being treated, e.g., the particular animal treated, age, weight, health, types of concurrent treatment, if any, and frequency of treatments, and can be easily determined by one of skill in the art (e.g., by the clinician). Those skilled in the art will appreciate that dosages may be determined with guidance, for example, from Goodman & Goldman's The Pharmacological Basis of Therapeutics, Ninth Edition (1996), Appendix II, pp. 1707-1711 or from Goodman & Goldman's The Pharmacological Basis of Therapeutics, Tenth Edition (2001), Appendix II, pp. 475-493 both of which are hereby incorporated by reference in their entireties. With respect to conventional prenylation enzyme inhibitors, guidance may be obtained from art-recognized dosage amounts as described, for example, by J. E. Karp, et al., Blood, 97(11):3361-3369 (2001) and A. A. Adjei, et al., Cancer Research, 60:1871-1877 (2000) hereby incorporated by reference in its entirety.

In various embodiments, an effective amount of an activated fatty acid delivered during each administration cycle may range from about 10 mg/m²/day to about 1000 mg/m²/day. In some embodiments, an effective amount may be about 20 mg/m²/day to about 700 mg/m²/day, and in others, an effective amount may be about 30 mg/m²/day to about 600 mg/m²/day. In particular embodiments, an effective amount may be about 50 mg/m²/day, about 400 mg/m²/day, about 500 mg/m²/day, or about 600 mg/m²/day. In yet other embodiments, an effective amount of an activated fatty acid may vary as treatment progresses. For example, a dosage regimen may be increased or decreased as treatment proceeds through administration cycles, or the daily dosage may increase or decrease throughout administration. In additional embodiments, greater than 1000 mg/m²/day may be administered because even high doses of activated fatty acid are generally tolerable to the patient and may not produce undesired physiological effects.

In some embodiments, activated fatty acids administered may include up to at least 1% by weight up to at least 10% by weight, up to at least 20% by weight, at least 30% by weight, at least 40% by weight, at least 50% by weight, at least 60% by weight, at least 70% by weight, at least 80% by weight, at least 90% by weight or at least 100% by weight of one or more species of activated fatty acid. In particular embodiments, a single species of activated ω-3 fatty acid may make up at least 50%, at least 60% by weight, at least 70% by weight, at least 80% of the total activated fatty acid administered, and in other embodiments, a single species of activated omega-3 fatty acids may make up about 5 to about 100% by weight, about 25 to about 75% by weight, or about 40 to about 55% by weight of the fatty acids administered. In particular embodiments, the ratio of activated fatty acid to non-activated may be from about 99:1 to about 1:99, about 99:1 to about 90:10, about 90:10 to about 80:20, about 80:20 to about 70:30, about 70:30 to about 60:40, about 60:40 to about 50:50, about 50:50 to about 40:60, about 40:60 to about 30:70, about 30:70 to about 20:80, about 20:80 to about 10:90, about 10:90 to about 1:99 about 1:4 to about 4:1, about 1:3 to about 3:1 or about 1:2 to about 2:1.

In yet other embodiments activated fatty acids administered may include up to at least 0.01%, 0.025%, 0.05%, 0.1%, 0.5%, 1.0%, 10.0%, 20.0% and 30.0% by weight of one or more species of activated fatty acid.

For example, in some embodiments, the activated omega-3 fatty acids may be prepared from one of EPA or DHA or a combination of EPA and DHA. The composition administered may include about 5 to about 100% by weight, about 25 to about 75% by weight, or about 30 to about 60% by weight activated EPA and/or activated DHA, and any remainder may be made up of non-activated EPA and/or DHA. In compositions containing both activated EPA and activated DHA, the activated EPA and activated DHA may be present in a weight ratio of EPA:DHA of from 99:1 to 1:99, 1:4 to 4:1, 1:3 to 3:1 or 1:2 to 2:1. In compositions containing activated EPA and/or activated DHA as well as non-activated EPA and/or DHA, the weight ratio of activated:non-activated may be from 99:1 to 1:99, 99:1 to 90:10, 90:10 to 80:20, 80:20 to 70:30, 70:30 to 60:40, 60:40 to 50:50, 50:50 to 40:60, 40:60 to 30:70, 30:70 to 20:80, 20:80 to 10:90, 10:90 to 1:99, 1:4 to 4:1, 1:3 to 3:1 or 1:2 to 2:1. In the embodiments described above, the percentage by weight may be based on the free acid or ester forms, although it is preferably based on the ethyl ester form of the ω-3 fatty acids even if other forms are utilized in accordance with the present invention.

In another embodiment, the activated fatty acid can be prepared from conjugated fatty acids such as, for example (9Z,11E)-octadeca-9,11-dienoic acid and 10E, 12Z-octadeca-10,12-dienoic acid. The composition administered may include about 0.1 to about 100% by weight, about 25 to about 75% by weight, or about 30 to about 60% by weight activated (9Z,11E)-octadeca-9,11-dienoic acid and/or activated 10E, 12Z-octadeca-10,12-dienoic acid., and any remainder may be made up of non-activated (9Z,11E)-octadeca-9,11-dienoic acid and/or 10E, 12Z-octadeca-10,12-dienoic acid. In compositions containing activated (9Z,11E)-octadeca-9,11-dienoic acid and/or activated 10E, 12Z-octadeca-10,12-dienoic acid as well as non-activated 9Z,11E)-octadeca-9,11-dienoic acid and/or activated 10E, 12Z-octadeca-10,12-dienoic acid, the weight ratio of activated:non-activated may be from 99:1 to 1:99, 99:1 to 90:10, 90:10 to 80:20, 80:20 to 70:30, 70:30 to 60:40, 60:40 to 50:50, 50:50 to 40:60, 40:60 to 30:70, 30:70 to 20:80, 20:80 to 10:90, 10:90 to 1:99, 1:4 to 4:1, 1:3 to 3:1 or 1:2 to 2:1. In the embodiments described above, the percentage by weight may be based on the free acid or ester forms, although it is preferably based on the ethyl ester form of the ω-3 fatty acids even if other forms are utilized in accordance with the present invention.

In still other embodiments, the activated fatty acid may be prepared from a different base fatty acid than the non-activated fatty acids with which it is combined. For example, in some embodiments, the activated fatty acid may be an activated linoleic acid, an activated conjugated linoleic acid, an activated oleic acid, or combinations thereof, and these activated fatty acids may be combined with non-activated EPA and/or DHA. In such embodiments, the ratio of activated linoleic acid and/or activated oleic acid to non-activated EPA and/or DHA may be from about 99:1 to 1:99, 99:1 to 90:10, 90:10 to 80:20, 80:20 to 70:30, 70:30 to 60:40, 60:40 to 50:50, 50:50 to 40:60, 40:60 to 30:70, 30:70 to 20:80, 20:80 to 10:90, 10:90 to 1:99, 1:4 to 4:1, 1:3 to 3:1, 1:2 to 2:1, or 1:1. In particular embodiments, activated linoleic acid or oleic acid may be combined with EPA and DHA, and each of the three components may be provided in a ratio of from about 1:1:1, 2:1:1, 1:2:1, 1:1:2, 2:2:1, 1:2:2, 3:1:1, and the like.

In embodiments in which activated fatty acid are combined with a secondary form of treatment, the activated fatty acid may be administered in a separate dosage unit from the secondary agent such that each treatment is provided separately. In other embodiments, the activated fatty acid may be provided in the same dosage unit as one or more secondary agent. In such embodiments, the activated fatty acid may be combined with the one or more secondary agent in a range of about 1:1000 to about 1000:1, about 1:1000 to about 100:900, about 100:900 to about 200:800, about 200:800 to about 300:700, about 300:700 to about 400:600, about 400:600 to about 300:700, about 300:700 to about 200:800, about 200:800 to about 100:900, about 100:900 to about 1:1000 by weight or about 200:1 to about 200:1 by weight. For example, in some embodiments, the activated fatty acid may be present in an amount from about 1 mg to about 3000 mg or from about 10 mg to about 2000 mg, and the one or more secondary agents may be present in an amount from about 1 mg to about 1000 mg, about 5 mg to about 500 mg, and about 5 mg to about 100 mg.

In some embodiments, the dosage regimen as described above may be combined with a secondary form of treatment or a secondary agent. For example, activated fatty acids such as those described above may be combined with antioxidants, statins, squalene synthesis inhibitors, azetidinone-based compounds, LDL catabolism activators, PPAR antagonists or agonists, antiarrhythmic agent, NSAIDs and the like, and combinations thereof.

In particular embodiments, the activated fatty acids of the invention may be mixed with one or more nutraceutical equivalents to any of the agents described above. For example, in some embodiments, the activated fatty acids of the invention may be mixed with a nutraceutical statin equivalent such as, for example, from rice bran oil, enzyme-treated stabilized rice bran, a solubilized fraction of rice bran oil, and derivatives thereof and the like. In other embodiments, one or more nutraceutical including, but not limited to, glucosamine derivatives, methylsulfonylmethane, yucca concentrates, grape seed extracts, beta-carotene, ephedra, ginko biloba, goldenseal, valerian, ginseng, echinacea, and the like may be combined with activated fatty acids.

Embodiments further include nutraceuticals including the nutraceutical equivalents to any of the agents described above and one or more activated fatty acids. Thus, in certain embodiments, the nutraceuticals may include one or more activated fatty acid in combination with one or more other nutraceutical compound or one or more other secondary agent. Nutraceuticals containing various combinations of ingredients are well known in the art, and any known nutraceutical may be combined with one or more activated fatty acids to produce a combination nutraceutical. For example, in various embodiments, activated fatty acids may be combined with vitamins including vitamins A, B, including vitamin B-1, B-2, B-6, B-12, C, D including vitamin D3, and E, and the like and derivatives thereof, minerals such as selenium and the like, plant extracts such as β-carotene, ginko biloba, goldenseal, valerian, ginseng, echinacea, grape seed extracts, ephedra, yucca concentrates, green tea extract, rice bran extract, wheat germ, wheat germ extract, beeswax, red yeast rice extract, stevia leaf extract, and the like, nutraceutical oils such as flaxseed oil, borage seed oil, and other know nutraceutical components such as coenzyme Q10, glucosamine derivatives, methylsulfonylmethane, pantothenic acid, biotin, thiamin, riboflavin, niacin, folic acid, palmitic acid, and the like. Thus, without wishing to be bound by theory, nearly any nutraceutical can be incorporated into the activated fatty acid containing nutraceuticals described herein.

In particular embodiments, one or more additional ingredients may be provided to produce a nutraceutical for treating or preventing specific diseases or indication. For example, in some embodiments, activated fatty acids may be combined with other nutraceutically active components that can act as antioxidants such as vitamin C, vitamin E, vitamin D, selenium and the like to create a nutraceutical for treating aging and cancer. In other embodiments, a nutraceutical for treating or preventing diseases of the eye may be prepared by combining activated fatty acids with, for example, vitamin A and/or β-carotene, and in still other embodiments, a nutraceutical with neuroprotective activities or that enhances cognitive abilities may be prepared by combining activated fatty acids with, for example, ginko biloba. In yet other embodiments, nutraceuticals for treating or preventing heart or circulatory diseases may be prepared by combining activated fatty acids with policosanol, guggulipids, rice bran extract, enzyme-treated stabilized rice bran, a solubilized fraction of rice bran oil, wheat germ, wheat germ extract, beeswax, red yeast rice extract, and or other nutraceuticals known to exhibit statin-like activity. In further embodiments, components with various activities may be combined. For example, a nutraceutical with neuroprotective activities may include one or more antioxidants such as vitamin C, vitamin E, or selenium along with ginko biloba, since it is well known that antioxidants are also effective neuroprotectants. In yet other embodiments, vitamin E may be provided to any nutraceutical described herein to stabilize the activated fatty acids and increase the shelf life of the nutraceutical.

Nutraceuticals having fatty acids and one or more additional nutraceutically active components may be combined in a single dose formulation by known methods. For example, in some embodiments, lipophilic additional nutraceutically active components may be combined with the activated fatty acids directly. In other embodiments, the activated fatty acid may be separated from a non-lipophilic additional nutraceutically active component by, for example, preparing separate cores that are combined into a single capsule or incorporating the non-lipophilic additional nutraceutically active component into one or more coating layers.

In embodiments in which activated fatty acid are combined with a secondary form of treatment, the activated fatty acid may be administered in a separate dosage unit from the secondary agent such that each treatment is provided separately. In other embodiments, the activated fatty acid may be provided in the same dosage unit as one or more secondary agent. In such embodiments, the activated fatty acid may be combined with the one or more secondary agent in a range of about 1:1000 to about 1000:1, about 1:1000 to about 100:900, about 100:900 to about 200:800, about 200:800 to about 300:700, about 300:700 to about 400:600, about 400:600 to about 300:700, about 300:700 to about 200:800, about 200:800 to about 100:900, about 100:900 to about 1:1000 by weight or about 200:1 to about 200:1 by weight. For example, in some embodiments, the activated fatty acid may be present in an amount from about 1 mg to about 3000 mg or from about 10 mg to about 2000 mg, and the one or more secondary agents may be present in an amount from about 1 mg to about 1000 mg, about 5 mg to about 500 mg, and about 5 mg to about 100 mg.

In certain embodiments, a single dosage unit may include about 500 mg to about 2000 mg or about 1000 mg of one or more activated ω-3 fatty acids, and about 1 mg to about 150 mg or about 5 mg to about 100 mg of a statin compound, about 1 mg to about 300 mg or 10 to about 100 mg of a fibrate compound or a combination thereof.

In other embodiments, a single dosage unit may include about 500 mg to about 2000 mg or about 1000 mg of one or more activated ω-7 fatty acids, and about 1 mg to about 150 mg or about 5 mg to about 100 mg of a statin compound, about 1 mg to about 300 mg or 10 to about 100 mg of a fibrate compound or a combination thereof.

The activated fatty acids of various embodiments may be prepared by any method known in the art. For example, in particular embodiments, the activated fatty acids may be derived from natural sources such as, for example, fish oils which may contain activated fatty acids, and in particular, nitro-fatty acids, that can be isolated, purified or concentrated form the fish oil. In other embodiments, an activated fatty acid may be prepared by contacting an naturally occurring unsaturated fatty acids with one or more nitro containing compounds or nitrogenating agents. Such naturally occurring activated fatty acids may be useful in the production of nutraceuticals.

In other embodiments, the method may be carried out in the presence of one or more cofactors and/or catalysts. For example, in certain embodiments, activated fatty acids may be prepared by combining an unsaturated fatty acid with a nitrogenating agent such as ammonia or primary amines, molecular oxygen and an oxidation catalyst as described in U.S. Pat. No. 4,599,430, which is hereby incorporated by reference in its entirety.

Methods for preparing activated fatty acids are incorporated by reference from U.S. application 61/141,844 and US2010/01669218 A1.

Embodiments of the invention also include topical compositions containing activated fatty acids and, in some embodiments, one or more secondary agents and/or non-activated fatty acids. For example, in some embodiments, the topical composition may include one or more activated fatty. In such embodiments, the one or more activated fatty acids may comprise about 10% by weight to about 95% by weight of the total composition. In other embodiments activated fatty acids comprise about 0.01% to about 10% by weight of one or more species of activated fatty acid. In yet other embodiments activated fatty acids comprise about 95% to about 100% by weight of one or more species of activated fatty acid.

In some embodiments, topical compositions of the invention can contain additional ingredients commonly found in skin care compositions and cosmetics, such as for example, tinting agents, emollients, skin conditioning agents, emulsifying agents, humectants, preservatives, antioxidants, perfumes, chelating agents etc., that are compatible with other components of the composition.

As nitroalkenes are very reactive molecules a nitroalkene topical composition desirably includes a substantial antioxidant and preservative system. In one preferred embodiment, the antioxidant system is Oxynex™ AP, Oynex™ LM, or Oxynex™ K. The preferred embodiments use fatty acids of Vitamin C, specifically ascorbyl palmitate, as a significant component of the antioxidant system. Antioxidants are typically present in an amount ranging from about 0.025% to about 5.00% by weight of the composition, include, but are not limited to, butylated hydroxy toluene (BHT); vitamin C and/or vitamin C derivatives, such as fatty acid esters of ascorbic acid, particularly asocorbyl palmitate; butylated hydroanisole (BHA); phenyl-a-naphthylamine; hydroquinone; propyl gallate; nordihydroquiaretic acid; vitamin E and/or derivatives of vitamin E, including tocotrienol and/or tocotrienol derivatives; calcium pantothenates; green tea extracts; mixed polyphenols; and mixtures of any of these. As mentioned above, particularly preferred antioxidants are those that provide additional benefits to the skin such as ascorbyl palmitate. Preservatives are typically present in an amount ranging from about 0.5% to about 2.0% by weight percent, based on the total composition.

Emollients, typically present in amounts ranging from about 0.01% to 5% of the total composition include, but are not limited to, fatty esters, fatty alcohols, mineral oils, polyether siloxane copolymers, and mixtures thereof. Humectants may be present in amounts ranging from about 0.1% to about 5% by weight of the total composition. Non-polar humectants are preferred. Emulsifiers, typically present in amounts from about 1% to about 10% by weight of the composition, include, but are not limited to, stearic acid, cetyl alcohol, stearyl alcohol, steareth 2, steareth 20, acrylates/C 10-30 alkyl acrylate crosspolymers, and mixtures thereof. Chelating agents, typically present in amounts ranging from about 0.01% to about 2% by weight, include, but are not limited to, ethylenediamine tetraacetic acid (EDTA) and derivatives and salts thereof, dihydroxyethyl glycine, tartaric acid, and mixtures thereof.

Some embodiments of this invention contain at least one other adjunct ingredient in addition to nitroalkene(s). Fat-soluble fatty acid esters of ascorbic acid (vitamin C) are employed as an adjunct ingredient as well as an antioxidant in some embodiments. The more oxidation-resistant saturated fatty acid esters of ascorbic acid are preferred, including, but not limited to, ascorbyl laurate, ascorbyl myristate, ascorbyl palmitate, ascorbyl stearate, and ascorbyl behenate. Ascorbyl palmitate is used in one preferred embodiment. Other possible adjunct ingredients include, but are not limited to one or more of: amino acids, lipoic acid; or tocotrienols and tocotrienol derivatives and vitamin E compositions enriched with tocotrienols or tocotrienol derivatives

Additional ingredients and methods disclosed in U.S. Pat. Nos. 4,775,530, 5,376,361, 5,409,693, 5,545,398, 5,574,063, 5,643,586, 5,709,868, 5,879,690, 5,965,618, 6,051,244, 6,162,419, and 6,191,121 are hereby incorporated by reference to the extent that they support the present specification.

In particular embodiments, the one or more activated fatty acids may be mixed with one or more stabilizers such as, for example, antioxidants, vitamin E, vitamin C, β-carotene, wheat germ oil and the like, and in some embodiments, the one or more activated fatty acid contained in the composition may be combined with one or more solubilizers such as, for example, surfactants, hydrophilic or hydrophobic solvents, oils or combinations thereof.

For example, in some embodiments a solubilizer may be vitamin E or a vitamin E derivative such as, but not limited to, α-, β-, γ-, δ-, ζ1-, ζ2- and ε-tocopherols, their dI, d and I forms and their structural analogues, such as tocotrienols; the corresponding derivatives, esters, produced with organic acids; and mixtures thereof. In particular embodiments, vitamin E derivative solubilizers may include tocopherols, tocotrienols and tocopherol derivatives with organic acids such as acetic acid, propionic acid, bile acid, lactic acid, pyruvic acid, oxalic acid, malic acid, malonic acid, succinic acid, maleic acid, fumaric acid, tartaric acid, citric acid, benzoic acid, cinnamic acid, mandelic acid, polyethylene glycol succinate and salicylic acid.

In other embodiments, monohydric alcohol including, for example, ethanol, isopropanol, t-butanol, a fatty alcohol, phenol, cresol, benzyl alcohol or a cycloalkyl alcohol, or monohydric alcohol esters of organic acids such as, for example, acetic acid, propionic acid, butyric acid, a fatty acid of 6-22 carbon atoms, bile acid, lactic acid, pyruvic acid, oxalic acid, malic acid, malonic acid, succinic acid, maleic acid, fumaric acid, tartaric acid, citric acid, benzoic acid, cinnamic acid, mandelic acid and salicylic acid may be used as solubilizers. In certain embodiments, solubilizers in this group may include trialkyl citrates such as triethyl citrate, acetyltriethyl citrate, tributyl citrate, acetyltributyl citrate and mixtures thereof; lower alcohol fatty acid esters such as ethyl oleate, ethyl linoleate, ethyl caprylate, ethyl caprate, isopropyl myristate, isopropyl palmitate and mixtures thereof and lactones ε-caprolactone, δ-valerolactone, β-butyrolactone, isomers thereof and mixtures thereof.

In still other embodiments, the solubilizer may be a nitrogen-containing solvent such as, for example, dimethylformamide, dimethylacetamide, N-alkylpyrrolidone, N-hydroxyalkylpyrrolidone, N-alkylpiperidone, N-alkylcaprolactam and mixtures thereof wherein alkyl may be a C₁₋₁₂ branched or straight chain alkyl. In particular embodiments, nitrogen-containing solvents may include N-methyl 2-pyrrolidone, N-ethyl 2-pyrrolidone or a mixture thereof. Alternatively, the nitrogen-containing solvent may be in the form of a polymer such as polyvinylpyrrolidone.

In yet other embodiments, solubilizers may include phospholipids such as phosphatidylcholine, phosphatidylethanolamine, phosphatidylserine, phosphatidylinositol, lecithins, lysolecithins, lysophosphatidylcholine, polyethylene glycolated phospholipids/lysophospholipids, lecithins/lysolecithins and mixtures thereof.

In still other embodiments, glycerol acetates and acetylated glycerol fatty acid esters and glycerol fatty acid esters may be used as solubilizers. In such embodiments, glycerol acetates may include acetin, diacetin, triacetin and mixtures thereof. Acetylated glycerol fatty acid esters may include acetylated monoglycerides, acetylated diglycerides and mixtures thereof with a fatty acid component that may be about 6 to about 22 carbon atoms. Glycerol fatty acid ester may be a monoglyceride, diglyceride, triglyceride, medium chain monoglycerides with fatty acids having about 6-12 carbons, medium chain diglycerides with fatty acids having about 6-12 carbons, medium chain triglycerides with fatty acids having about 6-12 carbons and mixtures thereof.

Further embodiments include solubilizers that may be a propylene glycol esters or ethylene glycol esters. In such embodiments, propylene glycol esters may include, for example, propylene carbonate, propylene glycol monoacetate, propylene glycol diacetate, propylene glycol fatty acid esters, acetylated propylene glycol fatty acid esters and mixtures thereof. Alternatively, propylene glycol fatty acid esters may be a propylene glycol fatty acid monoester, propylene glycol fatty acid diester or mixture thereof. In certain embodiments, propylene glycol ester may be propylene glycol monocaprylate, propylene glycol dicaprylate, propylene glycol dicaprate, propylene glycol dicaprylate/dicaprate and mixtures thereof. Ethylene glycol esters may include monoethylene glycol monoacetates, diethylene glycol esters, polyethylene glycol esters, ethylene glycol monoacetates, ethylene glycol diacetates, ethylene glycol fatty acid monoesters, ethylene glycol fatty acid diesters, polyethylene glycol fatty acid monoesters, polyethylene glycol fatty acid diesters and mixtures thereof. In such embodiments, the fatty acid may have about 6 to about 22 carbon atoms.

Hydrophilic solvents may also be utilized as solubilizers include, for example, alcohols, for example, water miscible alcohols, such as, ethanol or glycerol; glycols such as 1,2-propylene glycol; polyols such as a polyalkylene glycol, for example, polyethylene glycol. Alternatively, hydrophilic solvents may include N-alkylpyrolidones such as N-methylpyrolidone, triethylcitrate, dimethylisosorbide, caprylic acid or propylene carbonate.

The topical compositions are based on a carrier in which the nitroalkene is soluble per se or is effectively solubilized (e.g. as an emulsion or microemulsion). The carrier is dermatologically acceptable in the sense of not bringing about any adverse effect on the skin areas to which it is applied. The carrier preferably is appropriately selected for topical application, and forms a film or layer on the skin to which it is applied so as to localize the application. The nitroalkene is applied in admixture with the dermatologically acceptable carrier or vehicle (e.g. as a lotion, cream, gel, ointment, soap, stick, or the like) to as to facilitate topical application and provide therapeutic effects.

Non-polar and hydrophobic carriers are required for the compositions of the invention. Aqueous solvents and other polar solvents should be avoided because nitroalkenes are unstable in such solvents. Carriers may include polyethylene glycol, including PEG-1000, PEG-200, PEG-400; PEG-600; Labrasol® (a lipid-based self-emulsifying excipient mainly composed of PEG esters and glycerides with medium acyl chains); glycerin; polypropylene glycol; Stabileze® 06 (a PVM/MA Decadiene Crosspolymer); hydrogenated polyisobutane/polyethane; Permethyl® 99A (isododecane); BV-OSC (tetrahexyldecyl ascorbate); VC-IP (tetrahexyldecyl ascorbate); Vitamine E; beta carotene; disopropyl adipate; 2-ethylhexyl pentate; oleth-3; Ceraphyl® 31 (Propanoic acid 2-hydroxy-dodecyl ester); Ceraphyl® 41 (Propanoic acid, 2-hydroxy-, C12-15-alkyl esters); Glycereth-4; Glycereth-7; diglycerin; panthenol; and phytantriaol. Carrier formulations based principally on polymer polyethers such as polyethylene glycol and polypropylene glycol are a preferred embodiment.

A phosphatidycholine based carrier is another possible embodiment. Phosphatidylcholine, commonly called lecithin, is a mixture of diglycerides of stearic, palmitic, and oleic acids, linked to the choline ester of phosphoric acid. It can be isolated from eggs, soybeans, and other biological materials, chemically synthesized, or obtained commercially from many sources. Carrier formulations as disclosed in U.S. Pat. No. 7,182,956, the disclosure of which is hereby incorporated by reference, including polyenylphosphatidycholine enriched phosphatidycholine and polyglycol mixtures, are particularly preferred.

Additionally, in various embodiments, the activated fatty acid and/or one or more secondary agents of the invention may be formulated with one or more additional non-pharmaceutically active ingredients including, but not limited to, solubilizers, antioxidants, chelating agents, buffers, emulsifiers, thickening agents, dispersants, and preservatives.

Embodiments may also include film forming materials and/or binders and/or other conventional additives such as lubricants, fillers, antiadherents, antioxidants, buffers, solubilizers, dyes, chelating agents, disintegrants, and/or absorption enhancers. Surfactants may act as both solubilizers and absorption enhancers. Additionally, coatings may be formulated for immediate release, delayed or enteric release, or sustained release in accordance with methods well known in the art. Conventional coating techniques are described, e.g., in Remington's Pharmaceutical Sciences, 18th Ed. (1990), hereby incorporated by reference.

In some embodiments, a topical composition may also include one or more preservatives, coloring and opacifying agents, or combinations thereof. Suitable preservatives and colorants are known in the art and include, for example, benzoic acid, para-oxybenzoate, caramel colorant, gardenia colorant, carotene colorant, tar colorant and the like.

In embodiments in which one or more secondary agents are applied in a composition, the secondary agent may be provided as a homogenous solution or a heterologous suspension in a pharmaceutically acceptable solvent. Such pharmaceutically acceptable solvents may be an aqueous or organic solvent such as, for example, methanol, ethanol, isopropanol, ethylene glycol, acetone, or mixtures thereof. In other embodiments, pharmaceutically acceptable solvents may include, but are not limited to, polypropylene glycol; polypropylene glycol; polyethylene glycol, for example, polyethylene glycol 600, polyethylene glycol 900, polyethylene glycol 540, polyethylene glycol 1450, polyethylene glycol 6000, polyethylene glycol 8000, and the like; pharmaceutically acceptable alcohols that are liquids at about room temperature, for example, propylene glycol, ethanol, 2-(2-ethoxyethoxy)ethanol, benzyl alcohol, glycerol, polyethylene glycol 200, polyethylene glycol 300, polyethylene glycol 400 and the like; polyoxyethylene castor oil derivatives, for example, polyoxyethyleneglycerol triricinoleate or polyoxyl 35 castor oil, polyoxyethyleneglycerol oxystearate, RH 40 (polyethyleneglycol 40 hydrogenated castor oil) or RH 60 (polyethyleneglycol 60 hydrogenated castor oil), and the like; saturated polyglycolized glycerides; polyoxyethylene alkyl ethers, for example, cetomacrogol 1000 and the like; polyoxyethylene stearates, for example, PEG-6 stearate, PEG-8 stearate, polyoxyl 40 stearate NF, polyoxyethyl 50 stearate NF, PEG-12 stearate, PEG-20 stearate, PEG-100 stearate, PEG-12 distearate, PEG-32 distearate, PEG-150 distearate and the like; ethyl oleate, isopropyl palmitate, isopropyl myristate and the like; dimethyl isosorbide; N-methylpyrrolidinone; parafin; cholesterol; lecithin; suppository bases; pharmaceutically acceptable waxes, for example, carnauba wax, yellow wax, white wax, microcrystalline wax, emulsifying wax and the like; pharmaceutically acceptable silicon fluids; sorbitan fatty acid esters such as sorbitan laurate, sorbitan oleate, sorbitan palmitate, sorbitan stearate and the like; pharmaceutically acceptable saturated fats or pharmaceutically acceptable saturated oils, for example, hydrogenated castor oil (glyceryl-tris-12-hydroxystearate), cetyl esters wax (a mixture of primarily C₁₄-C₁₈ saturated esters of C₁₄-C₁₈ saturated fatty acids having a melting range of about 43-47° C.), glyceryl monostearate and the like.

Other pharmaceutical formulations containing the compounds of the invention and a suitable carrier can be in various forms including, but not limited to, solids, solutions, powder, fluid emulsions, fluid suspensions, semi-solids, and dry powders including an effective amount of an activated fatty acid of the invention. It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, antioxidants, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's, The Pharmaceutical Basis of Therapeutics, 6th Edition, MacMillan Publishing Co., New York (1980) both of which are hereby incorporated by reference in their entireties can be consulted.

The compounds of the present invention can be formulated for parenteral or intravenous administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids diluents such as oleic acid find use in the preparation of injectables. Additional fatty acids diluents that may be useful in embodiments of the invention include, for example, one or more of stearic acid, metallic stearate, sodium stearyl fumarate, fatty acid, fatty alcohol, fatty acid ester, glyceryl behenate, mineral oil, vegetable oil, paraffin, leucine, silica, silicic acid, talc, propylene glycol fatty acid ester, polyethoxylated castor oil, polyethylene glycol, polypropylene glycol, polyalkylene glycol, polyoxyethylene-glycerol fatty ester, polyoxyethylene fatty alcohol ether, polyethoxylated sterol, polyethoxylated castor oil, polyethoxylated vegetable oil, and the like. In some embodiments, the fatty acid diluent may be a mixture of fatty acids. In some embodiments, the fatty acid may be a fatty acid ester, a sugar ester of fatty acid, a glyceride of fatty acid, or an ethoxylated fatty acid ester, and in other embodiments, the fatty acid diluent may be a fatty alcohol such as, for example, stearyl alcohol, lauryl alcohol, palmityl alcohol, palmitoyl acid, cetyl alcohol, capryl alcohol, caprylyl alcohol, oleyl alcohol, linolenyl alcohol, arachidonic alcohol, behenyl alcohol, isobehenyl alcohol, selachyl alcohol, chimyl alcohol, and linoleyl alcohol and the like and mixtures thereof.

Among the acceptable vehicles and solvents that may be employed in a formulation are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids diluents such as oleic acid find use in the preparation of injectables. Additional fatty acids diluents that may be useful in embodiments of the invention include, for example, one or more of stearic acid, metallic stearate, sodium stearyl fumarate, fatty acid, fatty alcohol, fatty acid ester, glyceryl behenate, mineral oil, vegetable oil, paraffin, leucine, silica, silicic acid, talc, propylene glycol fatty acid ester, polyethoxylated castor oil, polyethylene glycol, polypropylene glycol, polyalkylene glycol, polyoxyethylene-glycerol fatty ester, polyoxyethylene fatty alcohol ether, polyethoxylated sterol, polyethoxylated castor oil, polyethoxylated vegetable oil, and the like. In some embodiments, the fatty acid diluent may be a mixture of fatty acids. In some embodiments, the fatty acid may be a fatty acid ester, a sugar ester of fatty acid, a glyceride of fatty acid, or an ethoxylated fatty acid ester, and in other embodiments, the fatty acid diluent may be a fatty alcohol such as, for example, stearyl alcohol, lauryl alcohol, palmityl alcohol, palmitoyl acid, cetyl alcohol, capryl alcohol, caprylyl alcohol, oleyl alcohol, linolenyl alcohol, arachidonic alcohol, behenyl alcohol, isobehenyl alcohol, selachyl alcohol, chimyl alcohol, and linoleyl alcohol and the like and mixtures thereof.

Other embodiments of the invention include activated fatty acid prepared as described above which are formulated as a solid dosage form for oral administration including capsules, tablets, pills, powders, and granules. In such embodiments, the active compound may be admixed with one or more inert diluent such as sucrose, lactose, or starch. Such dosage forms may also comprise, as in normal practice, additional substances other than inert diluents, e.g., lubricating agents such as magnesium stearate. In the case of capsules, tablets, and pills, the dosage forms may also comprise buffering agents and can additionally be prepared with enteric coatings.

Preparation of an activated fatty acid in solid dosage form may vary. For example, in one embodiment, a liquid or gelatin formulation of the activated fatty acid may be prepared by combining the activated fatty acid with one or more fatty acid diluent, such as those described above, and adding a thickening agent to the liquid mixture to form a gelatin. The gelatin may then be encapsulated in unit dosage form to form a capsule. In another exemplary embodiment, an oily preparation of an activated fatty acid prepared as described above may be lyophilized to for a solid that may be mixed with one or more pharmaceutically acceptable excipient, carrier or diluent to form a tablet, and in yet another embodiment, the activated fatty acid of an oily preparation may be crystallized to from a solid which may be combined with a pharmaceutically acceptable excipient, carrier or diluent to form a tablet.

Further embodiments which may be useful for oral administration of activated fatty acids include liquid dosage forms. In such embodiments, a liquid dosage may include a pharmaceutically acceptable emulsion, solution, suspension, syrup, and elixir containing inert diluents commonly used in the art, such as water. Such compositions may also comprise adjuvants, such as wetting agents, emulsifying and suspending agents, and sweetening, flavoring, and perfuming agents.

In still further embodiments, activated fatty acids of the invention can be formulated as a depot preparation. Such long acting formulations can be administered by implantation (for example, subcutaneously or intramuscularly) or by intramuscular injection. Depot injections can be administered at about 1 to about 6 months or longer intervals. Thus, for example, the compounds can be formulated with suitable polymeric or hydrophobic materials (for example, as an emulsion in an acceptable oil) or ion exchange resins, or as sparingly soluble derivatives, for example, as a sparingly soluble salt.

Other suitable diluents for formulations include, but are not limited to those described below:

Vegetable oil: As used herein, the term “vegetable oil” refers to a compound, or mixture of compounds, formed from ethoxylation of vegetable oil, wherein at least one chain of polyethylene glycol is covalently bound to the vegetable oil. In some embodiments, the fatty acids have between about twelve carbons to about eighteen carbons. In some embodiments, the amount of ethoxylation can vary from about 2 to about 200, about 5 to 100, about to about 80, about 20 to about 60, or about 12 to about 18 of ethylene glycol repeat units. The vegetable oil may be hydrogenated or unhydrogenated. Suitable vegetable oils include, but are not limited to castor oil, hydrogenated castor oil, sesame oil, corn oil, peanut oil, olive oil, sunflower oil, safflower oil, soybean oil, benzyl benzoate, sesame oil, cottonseed oil, and palm oil. Other suitable vegetable oils include commercially available synthetic oils such as, but not limited to, Miglyol™ 810 and 812 (available from Dynamit Nobel Chemicals, Sweden) Neobee™ M5 (available from Drew Chemical Corp.), Alofine™ (available from Jarchem Industries), the Lubritab™ series (available from JRS Pharma), the Sterotex™ (available from Abitec Corp.), Softisan™ 154 (available from Sasol), Croduret™ (available from Croda), Fancol™ (available from the Fanning Corp.), Cutina™ HR (available from Cognis), Simulsol™ (available from CJ Petrow), EmCon™ CO (available from Amisol Co.), Lipvol™ CO, SES, and HS-K (available from Lipo), and Sterotex™ HM (available from Abitec Corp.). Other suitable vegetable oils, including sesame, castor, corn, and cottonseed oils, include those listed in R. C. Rowe and P. J. Shesky, Handbook of Pharmaceutical Excipients, (2006), 5th ed., which is incorporated herein by reference in its entirety. Suitable polyethoxylated vegetable oils, include but are not limited to, Cremaphor™ EL or RH series (available from BASF), Emulphor™ EL-719 (available from Stepan products), and Emulphor™ EL-620P (available from GAF).

Mineral oils: As used herein, the term “mineral oil” refers to both unrefined and refined (light) mineral oil. Suitable mineral oils include, but are not limited to, the Avatech™ grades (available from Avatar Corp.), Drakeol™ grades (available from Penreco), Sirius™ grades (available from Shell), and the Citation™ grades (available from Avater Corp.).

Castor oils: As used herein, the term “castor oil”, refers to a compound formed from the ethoxylation of castor oil, wherein at least one chain of polyethylene glycol is covalently bound to the castor oil. The castor oil may be hydrogenated or unhydrogenated. Synonyms for polyethoxylated castor oil include, but are not limited to polyoxyl castor oil, hydrogenated polyoxyl castor oil, microgolglyceroli ricinoleas, macrogolglyceroli hydroxystearas, polyoxyl 35 castor oil, and polyoxyl 40 hydrogenated castor oil. Suitable polyethoxylated castor oils include, but are not limited to, the Nikkol™ HCO series (available from Nikko Chemicals Co. Ltd.), such as Nikkol HCO-30, HC-40, HC-50, and HC-60 (polyethylene glycol-30 hydrogenated castor oil, polyethylene glycol-40 hydrogenated castor oil, polyethylene glycol-0 hydrogenated castor oil, and polyethylene glycol-60 hydrogenated castor oil, Emulphor™ EL-719 (castor oil 40 mole-ethoxylate, available from Stepan Products), the Cremophore™ series (available from BASF), which includes Cremophore RH40, RH60, and EL35 (polyethylene glycol-40 hydrogenated castor oil, polyethylene glycol-60 hydrogenated castor oil, and polyethylene glycol-35 hydrogenated castor oil, respectively), and the Emulgin® RO and HRE series (available from Cognis PharmaLine). Other suitable polyoxyethylene castor oil derivatives include those listed in R. C. Rowe and P. J. Shesky, Handbook of Pharmaceutical Excipients, (2006), 5th ed., which is incorporated herein by reference in its entirety.

Sterol: As used herein, the term “sterol” refers to a compound, or mixture of compounds, derived from the ethoxylation of sterol molecule. Suitable polyethoxylated sterols include, but are not limited to, PEG-24 cholesterol ether, Solulan™ C-24 (available from Amerchol); PEG-30 cholestanol, Nikkol™ DHC (available from Nikko); Phytosterol, GENEROL™ series (available from Henkel); PEG-25 phyto sterol, Nikkol™ BPSH-25 (available from Nikko); PEG-5 soya sterol, Nikkol™ BPS-5 (available from Nikko); PEG-10 soya sterol, Nikkol™ BPS-10 (available from Nikko); PEG-20 soya sterol, Nikkol™ BPS-20 (available from Nikko); and PEG-30 soya sterol, Nikkol™ BPS-30 (available from Nikko). As used herein, the term “PEG” refers to polyethylene glycol.

Polyethylene glycol: As used herein, the term “polyethylene glycol” or “PEG” refers to a polymer containing ethylene glycol monomer units of formula —O—CH2-CH2-. Suitable polyethylene glycols may have a free hydroxyl group at each end of the polymer molecule, or may have one or more hydroxyl groups etherified with a lower alkyl, e.g., a methyl group. Also suitable are derivatives of polyethylene glycols having esterifiable carboxy groups. Polyethylene glycols useful in the present invention can be polymers of any chain length or molecular weight, and can include branching. In some embodiments, the average molecular weight of the polyethylene glycol is from about 200 to about 9000. In some embodiments, the average molecular weight of the polyethylene glycol is from about 200 to about 5000. In some embodiments, the average molecular weight of the polyethylene glycol is from about 200 to about 900. In some embodiments, the average molecular weight of the polyethylene glycol is about 400. Suitable polyethylene glycols include, but are not limited to polyethylene glycol-200, polyethylene glycol-300, polyethylene glycol-400, polyethylene glycol-600, and polyethylene glycol-900. The number following the dash in the name refers to the average molecular weight of the polymer. In some embodiments, the polyethylene glycol is polyethylene glycol-400. Suitable polyethylene glycols include, but are not limited to the Carbowax™ and Carbowax™ Sentry series (available from Dow), the Lipoxol™ series (available from Brenntag), the Lutrol™ series (available from BASF), and the Pluriol™ series (available from BASF).

Propylene glycol fatty acid ester: As used herein, the term “propylene glycol fatty acid ester” refers to a monoether or diester, or mixtures thereof, formed between propylene glycol or polypropylene glycol and a fatty acid. Fatty acids that are useful for deriving propylene glycol fatty alcohol ethers include, but are not limited to, those defined herein. In some embodiments, the monoester or diester is derived from propylene glycol. In some embodiments, the monoester or diester has about 1 to about 200 oxypropylene units. In some embodiments, the polypropylene glycol portion of the molecule has about 2 to about 100 oxypropylene units. In some embodiments, the monoester or diester has about 4 to about 50 oxypropylene units. In some embodiments, the monoester or diester has about 4 to about 30 oxypropylene units. Suitable propylene glycol fatty acid esters include, but are not limited to, propylene glycol laurates: Lauroglycol™ FCC and 90 (available from Gattefosse); propylene glycol caprylates: Capryol™ PGMC and 90 (available from Gatefosse); and propylene glycol dicaprylocaprates: Labrafac™ PG (available from Gatefosse).

Stearoyl macrogol glyceride: Stearoyl macrogol glyceride refers to a polyglycolized glyceride synthesized predominately from stearic acid or from compounds derived predominately from stearic acid, although other fatty acids or compounds derived from other fatty acids may used in the synthesis as well. Suitable stearoyl macrogol glycerides include, but are not limited to, Gelucire® 50/13 (available from Gattefossé).

In some embodiments, the diluent component comprises one or more of mannitol, lactose, sucrose, maltodextrin, sorbitol, xylitol, powdered cellulose, microcrystalline cellulose, carboxymethylcellulose, carboxyethylcellulose, methylcellulose, ethylcellulose, hydroxyethylcellulose, methylhydroxyethylcellulose, starch, sodium starch glycolate, pregelatinized starch, a calcium phosphate, a metal carbonate, a metal oxide, or a metal aluminosilicate.

Exemplary excipients or carriers for use in solid and/or liquid dosage forms include, but are not limited to:

Sorbitol: Suitable sorbitols include, but are not limited to, PharroSorbidex E420 (available from Cargill), Liponic 70-NC and 76-NC (available from Lipo Chemical), Neosorb (available from Roquette), Partech SI (available from Merck), and Sorbogem (available from SPI Polyols).

Starch, sodium starch glycolate, and pregelatinized starch include, but are not limited to, those described in R. C. Rowe and P. J. Shesky, Handbook of Pharmaceutical Excipients, (2006), 5th ed., which is incorporated herein by reference in its entirety.

Disintegrant: The disintegrant may include one or more of croscarmellose sodium, carmellose calcium, crospovidone, alginic acid, sodium alginate, potassium alginate, calcium alginate, an ion exchange resin, an effervescent system based on food acids and an alkaline carbonate component, clay, talc, starch, pregelatinized starch, sodium starch glycolate, cellulose floc, carboxymethylcellulose, hydroxypropylcellulose, calcium silicate, a metal carbonate, sodium bicarbonate, calcium citrate, or calcium phosphate.

Still further embodiments of the invention include activated fatty acids administered in combination with other active such as, for example, adjuvants, protease inhibitors, or other compatible drugs or compounds where such combination is seen to be desirable or advantageous in achieving the desired effects of the methods described herein.

Some embodiments are directed to a dietary supplement including a fatty acid component enriched for one or more activated fatty acids fatty acids and a nutraceutically acceptable excipient. In some embodiments, the activated fatty acid may be derived from an omega-3 fatty acid, an omega-6 fatty acid, an omega-9 fatty acid, and combinations thereof. In other embodiments, the activated fatty acid may be a nitro-fatty acid or a keto-fatty acid, and in particular embodiments, the activated fatty acid may be conjugated nitro-linoleic acid, nitro-linoleic acid, nitro-α-linoleic acid, nitro-γ-linoleic acid, nitro-oleic acid, nitro-eicosapentaenoic acid, nitro-docosahexaenoic acid, keto-linoleic acid, keto-α-linoleic acid, keto-γ-linoleic acid, keto-oleic acid, keto-eicosapentaenoic acid, keto-docosahexaenoic acid, conjugated linoleic acid or a derivative or combination thereof. In still other embodiments, the dietary supplement may also include one or more of linoleic acid, α-linoleic acid, γ-linoleic acid, oleic acid, eicosapentaenoic acid (EPA), docosahexaenoic acid (DHA), or derivatives thereof. DHA and/or nitratel DHA are preferable for cognitive disorders. In some embodiment, the dietary supplement may further include one or more nutraceutical selected from vitamin A, vitamin B, vitamin B-1, vitamin B-2, vitamin B-6, vitamin B-12, vitamin C, vitamin D, vitamin D3, vitamin E, selenium, β-carotene, ginko biloba, goldenseal, valerian, ginseng, echinacea, grape seed extracts, ephedra, yucca concentrates, green tea extract, rice bran extract, wheat germ, wheat germ extract, beeswax, red yeast rice extract, stevia leaf extract, flaxseed oil, borage seed oil, coenzyme Q10, glucosamine derivatives, methylsulfonylmethane, pantothenic acid, biotin, thiamin, riboflavin, niacin, folic acid, palmitic acid, and derivatives thereof.

In certain embodiments, the dietary supplement may be a gel capsule, and in some embodiments, the one or more activated fatty acids may be about 5% by weight to about 95% by weight of the total gel capsule.

In particular embodiments, the dietary supplement may include a first fatty acid component enriched for one or more: activated fatty acid selected from conjugated nitro-linoleic acid, nitro-linoleic acid, keto-linoleic acid, nitro-oleic acid, and keto-oleic acid and a second fatty acid component having one or more non-activated fatty acid selected from conjugated linoleic acid, linoleic acid, α-linoleic acid, γ-linoleic acid, oleic acid, eicosapentaenoic acid (EPA), docosahexaenoic acid (DHA), or derivatives thereof, and in some embodiments, the dietary supplement may further include vitamin E or a derivative thereof. In other embodiments, the dietary supplement may include one or more secondary agent including but not limited to vitamin A, vitamin B, vitamin B-1, vitamin B-2, vitamin B-6, vitamin B-12, vitamin C, vitamin D, vitamin D3, vitamin E, selenium, β-carotene, ginko biloba, goldenseal, valerian, ginseng, echinacea, grape seed extracts, ephedra, yucca concentrates, green tea extract, rice bran extract, wheat germ, wheat germ extract, beeswax, red yeast rice extract, stevia leaf extract, flaxseed oil, borage seed oil, coenzyme Q10, glucosamine derivatives, methylsulfonylmethane, pantothenic acid, biotin, thiamin, riboflavin, niacin, folic acid, palmitic acid, and derivatives thereof. In some embodiments, the dietary supplement may include one or more secondary agent selected from policosanols, guggulipds, rice bran extract, wheat germ, wheat germ extract, beeswax, and red yeast rice extract, and such a dietary supplement may be formulated to promote a healthy heart and circulatory system. In other embodiments, the dietary supplement may include one or more secondary agent selected from vitamin B-1, vitamin B-2, vitamin B-6, vitamin B-12, vitamin C, vitamin D, vitamin D3, vitamin E, selenium, goldenseal, valerian, ginseng, and echinacea and such a dietary supplement may be formulated to promote healthy cell proliferation. In still other embodiments, the dietary supplement may include one or more secondary agent selected from vitamin A, vitamin C, vitamin E, and β-carotene, and such a dietary supplement may be formulated to promote healthy eyes. In yet other embodiments, the dietary supplement may include one or more secondary agent selected from vitamin A, vitamin C, vitamin E, selenium, ginko biloba, goldenseal, valerian, ginseng, echinacea, ephedra, green tea extract, and yucca concentrate, and such a dietary supplement may be formulated to promote cognitive health or formulated as a neuroprotectant.

In further embodiments, at least one of the one or more secondary agent may include one or more agents selected from solubilizers, stabilizers, colorants, plasticizers diluents, fillers, disintegrants, binders, lubricants, surfactants, hydrophobic vehicles, water soluble vehicles, emulsifiers, buffers, humectants, moisturizers, antioxidants, or preservatives or a combination thereof.

In certain embodiments, compositions be formulated to include about 10 mg to about 500 mg of one or more activated fatty acid and from about 10 mg to about 100 mg of vitamin C. In other embodiments, such compositions may be formulated to include about 10 mg to about 500 mg of one or more activated fatty acid and from about 2 mg to about 50 mg of vitamin E.

The compositions of various embodiments may further include one or more film forming materials and/or binders and/or other conventional additives such as lubricants, fillers, antiadherents, antioxidants, buffers, solubilizers, dyes, chelating agents, disintegrants, and/or absorption enhancers. Surfactants may act as both solubilizers and absorption enhancers. Additionally, coatings may be formulated for immediate release, delayed or enteric release, or sustained release in accordance with methods well known in the art. Conventional coating techniques are described, e.g., in Remington's Pharmaceutical Sciences, 18th Ed. (1990), hereby incorporated by reference. Additional coatings to be employed in accordance with the invention may include, but are not limited to, for example, one or more immediate release coatings, protective coatings, enteric or delayed release coatings, sustained release coatings, barrier coatings, and combinations thereof. In some embodiments, an immediate release coating may be used to improve product elegance as well as for a moisture barrier, and taste and odor masking. Rapid breakdown of the film in gastric media is important, leading to effective disintegration and dissolution.

In some embodiments, the compositions may include at least one or more secondary agent. For example, in some embodiments, at least one polymer, such as, but not limited to cellulose derivatives such as hydroxyethyl cellulose, hydroxypropyl cellulose, hydroxypropyl methylcellulose, polyvinylpyrrolidone, polyvinylpyrrolidone/vinyl acetate copolymer, ethyl cellulose aqueous dispersions and combinations thereof, preferably hydroxpropyl cellulose, ethyl cellulose, and mixtures thereof, may be added to the composition at a ratio of polymer to secondary agent of from about 1:20 to about 20:1 by weight or about 1:5 to about 10:1 by weight. In particular, where the amount of secondary agent is less than about 15 mg, the amount of polymer may be from about 1:2 to about 5:1 or from about 1:1 to about 4:1, and in embodiments where the amount of secondary agent is about 15 mg or more, the amount of polymer may be from about 1:4 to about 4:1 or about 1:3 to about 2:1.

In embodiments in which one or more secondary agents are included in the composition, the secondary agent may be provided as a homogenous solution or a heterologous suspension in a pharmaceutically acceptable solvent. Such pharmaceutically acceptable solvents may be an aqueous or organic solvent such as, for example, methanol, ethanol, isopropranol, ethylene glycol, acetone, or mixtures thereof. In other embodiments, pharmaceutically acceptable solvents may include, but are not limited to, polypropylene glycol; polypropylene glycol; polyethylene glycol, for example, polyethylene glycol 600, polyethylene glycol 900, polyethylene glycol 540, polyethylene glycol 1450, polyethylene glycol 6000, polyethylene glycol 8000, and the like; pharmaceutically acceptable alcohols that are liquids at about room temperature, for example, propylene glycol, ethanol, 2-(2-ethoxyethoxy)ethanol, benzyl alcohol, glycerol, polyethylene glycol 200, polyethylene glycol 300, polyethylene glycol 400 and the like; polyoxyethylene castor oil derivatives, for example, polyoxyethyleneglycerol triricinoleate or polyoxyl 35 castor oil, polyoxyethyleneglycerol oxystearate, RH 40 (polyethyleneglycol 40 hydrogenated castor oil) or RH 60 (polyethyleneglycol 60 hydrogenated castor oil), and the like; saturated polyglycolized glycerides; polyoxyethylene alkyl ethers, for example, cetomacrogol 1000 and the like; polyoxyethylene stearates, for example, PEG-6 stearate, PEG-8 stearate, polyoxyl 40 stearate NF, polyoxyethyl 50 stearate NF, PEG-12 stearate, PEG-20 stearate, PEG-100 stearate, PEG-12 distearate, PEG-32 distearate, PEG-150 distearate and the like; ethyl oleate, isopropyl palmitate, isopropyl myristate and the like; dimethyl isosorbide; N-methylpyrrolidinone; parafin; cholesterol; lecithin; suppository bases; pharmaceutically acceptable waxes, for example, carnauba wax, yellow wax, white wax, microcrystalline wax, emulsifying wax and the like; pharmaceutically acceptable silicon fluids; sorbitan fatty acid esters such as sorbitan laurate, sorbitan oleate, sorbitan palmitate, sorbitan stearate and the like; pharmaceutically acceptable saturated fats or pharmaceutically acceptable saturated oils, for example, hydrogenated castor oil (glyceryl-tris-12-hydroxystearate), cetyl esters wax (a mixture of primarily C₁₄-C₁₈ saturated esters of C₁₄-C₁₈ saturated fatty acids having a melting range of about 43-47° C.), glyceryl monostearate and the like.

Other embodiments are directed to a gel capsule including a core having a fatty acid component enriched for one or more activated fatty acids and one or more coating layers encapsulating the core. In some embodiments, the gel capsule may be flavored, and in particular embodiments, the flavoring agent may be a flavor selected from berry, strawberry, chocolate, cocoa, lemon, butter, almond, cashew, macadamia nut, coconut, blueberry, blackberry, raspberry, peach, lemon, lime, mint, orange, banana, chili pepper, pepper, cinnamon, and pineapple. In some embodiments, at least one of the one or more coating layers may include at least one flavoring agent, and in other embodiments, the core may include at least one flavoring agent. In further embodiments, at least one of the one or more coating layers may be an enteric coating, and in still further embodiments, the core may further include one or more agents selected from solubilizers, stabilizers, colorants, plasticizers diluents, fillers, disintegrants, binders, lubricants, surfactants, hydrophobic vehicles, water soluble vehicles, emulsifiers, buffers, humectants, moisturizers, antioxidants, or preservatives. In some embodiments, the core, at least one of the one or more coating layers, or a combination thereof further comprises one or more secondary agents.

In certain embodiments, such gel capsules may be formulated to include a core having from about 10 mg to about 500 mg of one or more activated fatty acid and from about 10 mg to about 100 mg of vitamin C and one or more coating layers encapsulating the core, and the core, at least one of the one or more coating layers, or combinations thereof may include from about 0.25% by weight to about 3.0% by weight of one or more flavoring agents. In other embodiments, such gel capsules may be formulated to include a core having from about 10 mg to about 500 mg of one or more activated fatty acid and from about 2 mg to about 50 mg of vitamin E and one or more coating layers encapsulating the core, and the core, at least one of the one or more coating layers, or combinations thereof may include from about 0.25% by weight to about 3.0% by weight of one or more flavoring agents.

The activated fatty acid containing core may be coated with one or more coating layer. For example, in some embodiments, the gel capsule may include a water-soluble gel layer between the coating layer and the activated fatty acid core. In other embodiments, the gel capsules may include a number of additional coatings on the capsules such as, for example, immediate release coatings, protective coatings, enteric or delayed release coatings, sustained release coatings, barrier coatings, and combinations thereof. In some embodiments, one or more secondary agent or non-activated fatty acid may be mixed with the activated fatty acid and/or be present in either a coating layer, a water-soluble gel layer, or an additional coating layer. Additionally, in various embodiments, the activated fatty acid and/or one or more secondary agents of the invention may be formulated with one or more additional non-pharmaceutically active ingredients including, but not limited to, solubilizers, antioxidants, chelating agents, buffers, emulsifiers, thickening agents, dispersants, and preservatives. In some embodiments, the activated fatty acids may be encapsulated in a coating prepared from gelatin as described in U.S. Pat. No. 6,531,150, which is hereby incorporated by reference in its entirety. The gelatin layer may further include one or more other non-gelatin protein and/or one or more polysaccharide such as, for example, albumin, pectin, guaran gum, carrageenan, agar and the like, and/or one or more additive such as, for example, enteric materials, plasticizers, preservatives, and the like. Enteric materials used in embodiments of the invention include any material that does not dissolve in the stomach when the gel capsule is administered orally and include, but are not limited to, pectin, alginic acid, cellulose such as carboxyl methylcellulose, celluloseacetate phthalate, and the like, Eudragit™, an acrylic copolymer. Without wishing to be bound by theory, the addition of an enteric coating may provide a means for masking the flavor of activated fatty acids by limiting the release of the activated fatty acids to the stomach. Plasticizers may include polyhydric alcohols, such as sorbitol, glycerin, polyethylene glycol and the like. In the embodiments described above, each coating layer may be from about 0.001 to about 5.00 mm or 0.01 to 1.00 mm thick.

The coatings of various embodiments may further include one or more film forming materials and/or binders and/or other conventional additives such as lubricants, fillers, antiadherents, antioxidants, buffers, solubilizers, dyes, chelating agents, disintegrants, and/or absorption enhancers. Surfactants may act as both solubilizers and absorption enhancers. Additionally, coating ma be formulated for immediate release, delayed or enteric release, or sustained release in accordance with methods well known in the art. Conventional coating techniques are described, e.g., in Remington's Pharmaceutical Sciences, 18th Ed. (1990), hereby incorporated by reference. Additional coatings to be employed in accordance with the invention may include, but are not limited to, for example, one or more immediate release coatings, protective coatings, enteric or delayed release coatings, sustained release coatings, barrier coatings, and combinations thereof. In some embodiments, an immediate release coating may be used to improve product elegance as well as for a moisture barrier, and taste and odor masking. Rapid breakdown of the film in gastric media is important, leading to effective disintegration and dissolution.

Capsular materials (i.e., the activated fatty acid containing core and/or one or more coating layers) may further include one or more preservatives, coloring and opacifying agents, flavorings and sweeteners, sugars, gastroresistant substances, or combinations thereof. Suitable preservative and colorant are known in the art and include, for example, benzoic acid, para-oxybenzoate, caramel colorant, gardenia colorant, carotene colorant, tar colorant and the like. In particular embodiments, one or more flavoring agents may be included the contents of the core of the gelatin capsule or in one or more coating layers of the capsule, or a combination thereof. For example, providing a palatable flavoring to the activated fatty acid gel capsule may be achieved by providing a flavored coating layer having a water soluble flavor. In such embodiments, from about 0.25% and about 1.50% by weight of said coating layer may be the water soluble flavoring. Any suitable flavor known in the art may be provided to the coating layer, such as, berry, strawberry, chocolate, cocoa, vanilla, lemon, nut, almond, cashew, macadamia nut, coconut, blueberry, blackberry, raspberry, peach, lemon, lime, mint, peppermint, orange, banana, chili pepper, pepper, cinnamon, and pineapple. In some embodiments, an oil soluble flavoring may be mixed with a activated fatty acid core that is encapsulated within the capsule. In such embodiments, from about 0.25% and about 1.50% by weight of said core may be the oil soluble flavoring. Such oil soluble flavoring may be similar to the taste of the flavor of the capsule, e.g., strawberry and strawberry, or the taste of the oil flavoring may be complementary to the capsule flavoring, e.g., banana and strawberry. Such flavoring agents and methods for providing flavoring to fatty acid containing capsules may be found in U.S. Pat. Nos. 6,346,231 and 6,652,879 which are hereby incorporated by reference in their entireties.

In some embodiments, the gel capsules of embodiments may include at least one coating layer including one or more secondary agent. In such embodiments, a layer including one or more secondary agent may be of sufficient thickness to prevent oxidative degradation of the one or more secondary agent. For example, in some embodiments, the thickness of this layer may be from about 5 to about 400 microns, about 10 to about 200 microns, about 20 to about 100 microns, or in certain embodiments, from about 40 to about 80 microns. In other embodiments, the thickness of such layers may be expressed in terms of percentage weight gain based on the total weight of the capsule. For example, a layer including one or more secondary agents may create a weight gain of about 0.05 to about 20%, about 0.1 to about 10%, about 0.1 to about 5%, and in particular embodiments about 0.25 to about 1%. In certain embodiments, a coating layer containing one or more secondary agent may further include at least one compound to prevent oxidative degradation. For example, in some embodiments, at least one polymer, such as, but not limited to cellulose derivatives such as hydroxyethyl cellulose, hydroxypropyl cellulose, hydroxypropyl methylcellulose, polyvinylpyrrolidone, polyvinylpyrrolidone/vinyl acetate copolymer, ethyl cellulose aqueous dispersions and combinations thereof, preferably hydroxpropyl cellulose, ethyl cellulose, and mixtures thereof, may be added to the coating layer at a ratio of polymer to secondary agent of from about 1:20 to about 20:1 by weight or about 1:5 to about 10:1 by weight. In particular, where the amount of secondary agent is less than about 15 mg, the amount of polymer may be from about 1:2 to about 5:1 or from about 1:1 to about 4:1, and in embodiments where the amount of secondary agent is about 15 mg or more, the amount of polymer may be from about 1:4 to about 4:1 or about 1:3 to about 2:1.

In embodiments in which one or more secondary agents are applied in a coating layer, the secondary agent may be provided as a homogenous coating solution or a heterologous suspension in a pharmaceutically acceptable solvent. Such pharmaceutically acceptable solvents may be an aqueous or organic solvent such as, for example, methanol, ethanol, isopropranol, ethylene glycol, acetone, or mixtures thereof. In other embodiments, pharmaceutically acceptable solvents may include, but are not limited to, polypropylene glycol; polypropylene glycol; polyethylene glycol, for example, polyethylene glycol 600, polyethylene glycol 900, polyethylene glycol 540, polyethylene glycol 1450, polyethylene glycol 6000, polyethylene glycol 8000, and the like; pharmaceutically acceptable alcohols that are liquids at about room temperature, for example, propylene glycol, ethanol, 2-(2-ethoxyethoxy)ethanol, benzyl alcohol, glycerol, polyethylene glycol 200, polyethylene glycol 30, polyethylene glycol 400 and the like, polyoxyethylene castor oil derivatives, for example, polyoxyethyleneglycerol triricinoleate or polyoxyl 35 castor oil, polyoxyethyleneglycerol oxystearate, RH 40 (polyethyleneglycol 40 hydrogenated castor oil) or RH 60 (polyethyleneglycol 60 hydrogenated castor oil), and the like; saturated polyglycolized glycerides; polyoxyethylene alkyl ethers, for example, cetomacrogol 1000 and the like; polyoxyethylene stearates, for example, PEG-6 stearate, PEG-8 stearate, polyoxyl 40 stearate NF, polyoxyethyl 50 stearate NF, PEG-12 stearate, PEG-20 stearate, PEG-100 stearate, PEG-12 distearate, PEG-32 distearate, PEG-150 distearate and the like; ethyl oleate, isopropyl palmitate, isopropyl myristate and the like; dimethyl isosorbide; N-methylpyrrolidinone; parafin; cholesterol; lecithin; suppository bases; pharmaceutically acceptable waxes, for example, carnauba wax, yellow wax, white wax, microcrystalline wax, emulsifying wax and the like; pharmaceutically acceptable silicon fluids; sorbitan fatty acid esters such as sorbitan laurate, sorbitan oleate, sorbitan palmitate, sorbitan stearate and the like; pharmaceutically acceptable saturated fats or pharmaceutically acceptable saturated oils, for example, hydrogenated castor oil (glyceryl-tris-12-hydroxystearate), cetyl esters wax (a mixture of primarily C₁₄-C₁₈ saturated esters of C₁₄-C₁₈ saturated fatty acids having a melting range of about 43-47° C.), glyceryl monostearate and the like.

Still other embodiments are directed to a method for preparing a gel capsule including the steps of combining gelswatch ingredients, melting the gelswatch ingredients to form a liquefied gelswatch, combining the liquefied gelswatch with a fatty acid component that is enriched for one or more activated fatty acids, and encapsulating the fatty acid component to form a gel capsule. In some embodiments, the method may further include drying the gel capsule, washing the gel capsule, and packaging the gel capsules. In certain embodiments, the gelswatch ingredients may include, for example, gelatin or a gelatin substitute, modified starch or other suitable gelatin substitute, a softener, glycerol, sorbitol or other suitable polyol, a flavoring agent, a coloring agent, keratin and combinations thereof.

Any method for preparing gel capsules known in the art may by used in various embodiments of the invention. For example, in one embodiment, capsules may be produced by a method including the steps of preparing a sheet of an outer coating layer and one or more sheets of other layers, laminating the sheets, drying the laminated sheets to obtain a dried sheet, and encapsulating one or more activated fatty acid or one or more activated fatty acids and one or more secondary agents within the dried sheet on a rotary filler to form a seamed capsule. In another embodiment, seamless capsules may be produced using an instrument equipped with two or more nozzles arranged concentrically. In other embodiments, gelatin capsules may be manufactured as, for example, a two-piece, sealed or unsealed hard gelatin capsule.

In another embodiment, a gelatin capsule including nitro fatty acids may be formed by the encapsulation of a dose of one or more nitro fatty acid in a gelatin capsule. In such embodiments, the gelatin capsule may be made of, for example, gelatin, glycerol, water, a flavoring, a coloring agent and combinations thereof, and the nitro fatty acid dose may be, for example, 180 mg of nitrated DHA and 60 mg of nitrated EPA. The manufacturing process of such embodiments may include the steps of combining gelswatch ingredients, melting and forming a liquefied gelswatch, delivering the liquefied gelswatch and the nitro fatty acid to an encapsulation machine, encapsulating a dose of nitro fatty acid, drying the encapsulated dose, washing the encapsulated dose and packaging the nitro fatty acid capsules for shipment. The gelswatch ingredients may include any ingredients described herein that are useful in the production of gelatin capsules such as, for example, gelatin or a gelatin substitute such as modified starch or other suitable gelatin substitute known in the art, a softener such as glycerol or sorbitol or other suitable polyol or other gelatin softener known in the art, a flavoring agent such as strawberry flavor Firmenich #52311A or other suitable gelatin capsule flavoring known in the art and optionally a coloring agent such as keratin or other suitable gelatin capsule coloring agent known in the art.

In particular embodiments, the gel capsule may be formed from a gelswatch mixture of about 45 parts by weight of gelatin, about 20 parts by weight of glycerol, about 35 parts by weight of water and about 0.5 or more parts by weight of flavoring. The gelswatch ingredients may be heated to about 60° C. to 70° C. and mixed together to form liquefied gelswatch. The liquefied gelswatch and the nitro fatty acid may then be poured into an encapsulation machine. The encapsulation machine then forms the nitro fatty acid capsule by encapsulating the nitro fatty acid dose into a gelatin capsule.

The capsule can then be dried at a temperature of, for example, about 20° C. The water content of the capsule may be reduced by evaporation during the drying step. The capsule can then be washed and ready for packaging, selling, or shipping. In some embodiments, a sweetener or flavoring agent can be added to the capsule through a dipping process. In the dipping process, the gelatin capsule is dipped in a sweetener/flavoring solution and then dried, allowing for the sweetener to form a coating around the outside of the capsule. In some embodiments, a sweetener or flavoring agent may be added to the capsule through an enteric coating process, and in other embodiments, a liquefied sweetener or flavoring agent can be sprayed on to the outside of the gelatin capsule and dried. Other methods of making gelatin capsules are known in the art and contemplated.

In various embodiments, the one or more coatings on the capsule may be applied by any technique known in the art including, but not limited to, pan coating, fluid bed coating or spray coating, and the one or more coatings may be applied, for example, as a solution, suspension, spray, dust or powder. For example, in some embodiments, a polymeric coating may be applied as aqueous-based solutions, organic-based solutions or dispersions containing and, in some embodiments, one or more secondary agent. In such embodiments, polymer-containing droplets may atomized with air or an inert gas and sprayed onto the core containing the activated fatty acids, and in some embodiments, heated air or inert gas may be added to facilitate evaporation of the solvent and film formation. In the case of soft gelatin capsules, the processing parameters of spray rate and bed temperature must be controlled to limit solubilization and capsule agglomeration. Additionally, a high bed temperature may result in evaporation of residual water from the capsule shell, causing the capsule to become brittle. In addition, coating uniformity which includes mass variance of the coated capsules and variance of the content of the coated activated fatty acid and accuracy of deposition must be evaluated.

Gel capsules of various embodiments of the invention may be of any shape such as, but not limited to, round, oval, tubular, oblong, twist off, or a non-standard shape (e.g., animal, tree, star, heart, etc.), and the size of the capsule may vary in accordance to the volume of the fill composition intended to be contained therein. For example, in some embodiments, hard or soft gelatin capsules may be manufactured using conventional methods as a single body unit comprising the standard capsule shape. A single-body soft gelatin capsule typically may be provided, for example, in sizes from 3 to 22 minims (1 minim=0.0616 ml) and in shapes of oval, oblong or others. Similarly, hard gel capsules may be manufactured using conventional methods in standard shapes and various standard sizes, such as those designated (000), (00), (0), (1), (2), (3), (4), and (5) where the largest number corresponds to the smallest size. Non-standard shapes may be used as well.

Other pharmaceutical formulations containing the compounds of the invention and a suitable carrier can be in various forms including, but not limited to, solids, solutions, powders, fluid emulsions, fluid suspensions, semi-solids, and dry powders including an effective amount of an activated fatty acid of the invention. It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, antioxidants, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's, The Pharmaceutical Basis of Therapeutics, 6th Edition, MacMillan Publishing Co., New York (1980) both of which are hereby incorporated by reference in their entireties can be consulted.

Other embodiments of the invention include activated fatty acid prepared as described above which are formulated as a solid dosage form for oral administration including capsules, tablets, pills, powders, and granules. In such embodiments, the active compound may be admixed with one or more inert diluent such as sucrose, lactose, or starch. Such dosage forms may also comprise, as in normal practice, additional substances other than inert diluents, e.g., lubricating agents such as magnesium stearate. In the case of capsules, tablets, and pills, the dosage forms may also comprise buffering agents and can additionally be prepared with enteric coatings.

Further embodiments are directed to methods for improving the health of an individual by administering to the individual a dietary supplement including a fatty acid component enriched for one or more activated fatty acids fatty acids, and a nutraceutically acceptable excipient. In some embodiments, the dietary supplement may include a first fatty acid component enriched for one or more activated fatty acid selected from nitro-linoleic acid, keto-linoleic acid, nitro-oleic acid, conjugated nitro-linoleic acid, and keto-oleic acid and a second fatty acid component having one or more non-activated fatty acid selected from linoleic acid, α-linoleic acid, γ-linoleic acid, oleic acid, eicosapentaenoic acid (EPA), docosahexaenoic acid (DHA), conjugated linoleic acid or derivatives thereof, and in particular embodiments, the dietary supplement may further include vitamin E or a derivative thereof. In some embodiments, the dietary supplement may further include one or more secondary agent selected from vitamin A, vitamin B, vitamin B-1, vitamin B-2, vitamin B-6, vitamin B-12, vitamin C, vitamin D, vitamin D3, vitamin E, selenium, β-carotene, ginko biloba, goldenseal, valerian, ginseng, echinacea, grape seed extracts, ephedra, yucca concentrates, green tea extract, rice bran extract, wheat germ, wheat germ extract, beeswax, red yeast rice extract, stevia leaf extract, flaxseed oil, borage seed oil, coenzyme Q10, glucosamine derivatives, methylsulfonylmethane, pantothenic acid, biotin, thiamin, riboflavin, niacin, folic acid, palmitic acid, and derivatives thereof. In some embodiments, the dietary supplement may include one or more secondary agent selected from policosanols, guggulipds, rice bran extract, wheat germ, wheat germ extract, beeswax, and red yeast rice extract, and such a dietary supplement may be formulated to promote a healthy heart and circulatory system. In other embodiments, the dietary supplement may include one or more secondary agent selected from vitamin B-1, vitamin B-2, vitamin B-6, vitamin B-12, vitamin C, vitamin D, vitamin D3, vitamin E, selenium, goldenseal, valerian, ginseng, and echinacea and such a dietary supplement may be formulated to promote healthy cell proliferation. In still other embodiments, the dietary supplement may include one or more secondary agent selected from vitamin A, vitamin C, vitamin E, and β-carotene, and such a dietary supplement may be formulated to promote healthy eyes. In yet other embodiments, the dietary supplement may include one or more secondary agent selected from vitamin A, Vitamin C, vitamin E, selenium, ginko biloba, goldenseal, valerian, ginseng, echinacea, ephedra, green tea extract, and yucca concentrate, and such a dietary supplement may be formulated to promote cognitive health or formulated as a neuroprotectant.

Various embodiments of the invention are also directed to compositions including one or more nitro fatty acids and one or more coating layers encapsulating the core. In such embodiments, the one or more nitro fatty acids may make up about 10% by weight to about 95% by weight of the total composition. As above, the compositions may include one or more additional secondary components such as, for example, rice bran oil, enzyme-treated stabilized rice bran, a solubilized fraction of rice bran oil, and derivatives thereof, glucosamine derivatives, methylsulfonylmethane, yucca concentrate, grape seed extract, beta-carotene, ephedra, ginko biloba, goldenseal, valerian, ginseng, green tea extract, and echinacea. The activated fatty acid may be derived from an omega-3 fatty acids, omega-6 fatty acids, omega-9 fatty acids, linoleic acid, conjugated linoleic acid, α-linoleic acid, oleic acid, eicosapentaenoic acid, docosahexaenoic acid or a derivative or combination thereof, and may contain non-activated fatty acids.

Various embodiments of the invention are also directed to compositions including a core having one or more nitro fatty acids and one or more coating layers encapsulating the core. In such embodiments, the one or more nitro fatty acids may make up about 10% by weight to about 95% by weight of the total gel capsule. As above, the compositions may include one or more additional secondary components such as, for example, rice bran oil, enzyme-treated stabilized rice bran, a solubilized fraction of rice bran oil, and derivatives thereof, glucosamine derivatives, methylsulfonylmethane, yucca concentrate, grape seed extract, beta-carotene, ephedra, ginko biloba, goldenseal, valerian, ginseng, green tea extract, and echinacea. The activated fatty acid may be derived from an omega-3 fatty acids, omega-6 fatty acids, omega-9 fatty acids, linoleic acid, α-linoleic acid, oleic acid, eicosapentaenoic acid, docosahexaenoic acid or a derivative or combination thereof, and may contain non-activated fatty acids.

Such compositions may be gel capsules, and such gel capsules may be flavored by providing one or more coating layers with at least one flavoring agent and/or the core with at least one flavoring agent. The flavoring agent may vary among embodiments and may be selected from berry, strawberry, chocolate, cocoa, lemon, butter, almond, cashew, macadamia nut, coconut, blueberry, blackberry, raspberry, peach, lemon, lime, mint, orange, banana, chili pepper, pepper, cinnamon, and pineapple, and in some embodiments, the gel capsule may an enteric coating. The core may further include other agents such as solubilizers, stabilizers, colorants, plasticizers diluents, fillers, disintegrants, binders, lubricants, surfactants, hydrophobic vehicles, water soluble vehicles, emulsifiers, buffers, humectants, moisturizers, antioxidants, preservatives or combinations thereof.

In still other embodiments, the core, at least one of the one or more coating layers, or a combination thereof may further include one or more secondary agents such as, for example, antioxidants, statins, squalene synthesis inhibitors, azetidinone-based compounds, low-density lipoprotein (LDL) catabolism activators, peroxisome proliferator-activated receptor (PPAR) antagonists or agonists, antiarrhythmic agent, non-steroidal anti-inflammatory drugs (NSAIDs) and nutraceutical equivalents thereof.

Embodiments of the invention also include methods for preparing a nitro-fatty acid by isolating nitro fatty acids from fish oils or plant oils, and methods for preparing a gel capsule by combining gelswatch ingredients; melting the gelswatch ingredients to form a liquefied gelswatch; combining the liquefied gelswatch with the nitro fatty acid; encapsulating the nitro fatty acid to form a gel capsule; drying the gel capsule; washing the gel capsule; and packaging the gel capsules.

Other methods for preparing a nitro fatty acid include the steps of contacting an existing unsaturated fatty acid composition with a nitro containing compound and reacting the existing unsaturated fatty acid with a nitro containing compound to form a nitro fatty acid. Methods for preparing a gel capsule including the steps of combining gelswatch ingredients, melting the gelswatch ingredients to form a liquefied gelswatch, combining the liquefied gelswatch with the nitro fatty acid, encapsulating the nitro fatty acid to form a gel capsule, drying the gel capsule, washing the gel capsule, and packaging the gel capsules.

Still other methods for preparing gel capsules including one or more activated fatty acid include the steps of contacting an unsaturated fatty acid with a mercuric salt and a selenium compound; contacting an intermediate resulting from step 1 with an electron withdrawing group donating reagent; reacting the intermediate resulting from step 2 with an oxidizing agent; combining gelswatch ingredients; melting the gelswatch ingredients to form a liquefied gelswatch; combining the liquefied gelswatch with the nitro fatty acid; encapsulating the nitro fatty acid to form a gel capsule; drying the gel capsule; washing the gel capsule; and packaging the gel capsules.

Yet other methods for preparing gel capsules including one or more activated fatty acid include the steps of combining a first component at least comprising an aliphatic hydrocarbon having an electron withdrawing group at one end and a second component at least comprising aliphatic hydrocarbon chain having an aldehyde at one end in the presence of a base to form a first intermediate; generating an alkene from the first intermediate; combining gelswatch ingredients; melting the gelswatch ingredients to form a liquefied gelswatch; combining the liquefied gelswatch with the nitro fatty acid; encapsulating the nitro fatty acid to form a gel capsule; drying the gel capsule; washing the gel capsule; and packaging the gel capsules.

Gelswatch ingredients may be selected from gelatin or a gelatin substitute, modified starch or other suitable gelatin substitute, a softener, glycerol, sorbitol or other suitable polyol, a flavoring agent, a coloring agent, keratin and combinations thereof.

This invention and embodiments illustrating the method and materials used may be further understood by reference to the following non-limiting examples.

### EXAMPLES

Although the present invention has been described in considerable detail with reference to certain preferred embodiments thereof, other versions are possible. Therefore the spirit and scope of the appended claims should not be limited to the description and the preferred versions contained within this specification. Various aspects of the present invention will be illustrated with reference to the following non-limiting examples.

### Examples 1-9

Exemplary compositions including gel capsules may be prepared as described above including the ingredients listed in Table 1.

| Com- | | | | | | | | | |
|---|---|---|---|---|---|---|---|---|---|
| pound | Ex. 1 | Ex. 2 | Ex. 3 | Ex. 4 | Ex. 5 | Ex. 6 | Ex. 7 | Ex. 8 | Ex. 9 |
| EPA¹ | 0 | 0 | 200 | 100 | 0 | 0 | 0 | 100 | 100 |
| DHA² | 400 | 400 | 200 | 300 | 180 | 360 | 140 | 100 | 100 |
| NO- | 0 | 200 | 100 | 0 | 0 | 0 | 0 | 0 | 400 |
| OLA³ | | | | | | | | | |
| NO- | 200 | 0 | 100 | 200 | 120 | 240 | 200 | 400 | 0 |
| LNA⁴ | | | | | | | | | |
| Vita- | 3.0 | 3.0 | 3.0 | 3.0 | 2.3 | 0 | 0 | 3.0 | 3.0 |
| min E | | | | | | | | | |
| Flavor- | | | | | 1.0 | 1.0 | 2.0 | 1.0 | 1.0 |
| ing | | | | | | | | | |

¹EPA—eicosapentaenoic acid
²DHA—docosahexaenoic acid
³OLA—oleic acid
⁴LNA—linoleic acid

### Example 10-20

Exemplary compositions including gel capsules may be prepared as described above including the ingredients listed in Table 2.

| | Ex. | Ex. | Ex. | Ex. | Ex. | Ex. | Ex. | Ex. | Ex. | Ex. |
|---|---|---|---|---|---|---|---|---|---|---|
| Compound | 10 | 12 | 13 | 14 | 15 | 16 | 17 | 18 | 19 | 20 |
| EPA¹ | 200 | 200 | 200 | 200 | 200 | 200 | 200 | 200 | 200 | 200 |
| DHA² | 200 | 200 | 200 | 200 | 200 | 200 | 200 | 200 | 200 | 200 |
| NO-OLA³ | 100 | 100 | 100 | 100 | 100 | 100 | 100 | 100 | 100 | 100 |
| NO-LNA⁴ | 100 | 100 | 100 | 100 | 100 | 100 | 100 | 100 | 100 | 100 |
| Vitamin E | 3 | 3 | 3 | 3 | 0 | 0 | 0 | 0 | 0 | 0 |
| Vitamin B12 | 20 | 20 | 0 | 0 | 20 | 20 | 0 | 0 | 0 | 20 |
| Folic Acid | 0 | 0.8 | 0 | 0 | 0 | 0.8 | 0 | 0 | 0 | 0.8 |
| Ginko Biloba | 0 | 0 | 400 | 0 | 0 | 0 | 400 | 0 | 400 | 400 |
| Ginseng | 0 | 0 | 0 | 200 | 0 | 0 | 0 | 200 | 200 | 200 |
| Flavoring | 2.0 | 2.0 | 2.0 | 2.0 | 2.0 | 2.0 | 2.0 | 2.0 | 2.0 | 2.0 |

¹EPA—eicosapentaenoic acid
²DHA—docosahexaenoic acid
³OLA—oleic acid
⁴LNA—linoleic acid

### Examples 21-29

Exemplary compositions including gel capsules may be prepared as described above including the ingredients listed in Table 3.

| Com- | Ex. | Ex. | Ex. | Ex. | Ex. | Ex. | Ex. | Ex. | Ex. |
|---|---|---|---|---|---|---|---|---|---|
| pound | 21 | 22 | 23 | 24 | 25 | 26 | 27 | 28 | 29 |
| EPA¹ | 0 | 0 | 200 | 100 | 0 | 0 | 0 | 100 | 100 |
| DHA² | 400 | 400 | 200 | 300 | 180 | 360 | 140 | 100 | 100 |
| NO- | 0 | 200 | 100 | 0 | 0 | 0 | 0 | 0 | 400 |
| OLA³ | | | | | | | | | |
| NO- | 200 | 0 | 100 | 200 | 120 | 240 | 200 | 400 | 0 |
| LNA⁴ | | | | | | | | | |
| NO- | 200 | 200 | 100 | 200 | 120 | 240 | 200 | 400 | 400 |
| CLA⁵ | | | | | | | | | |
| Vita- | 3.0 | 3.0 | 3.0 | 3.0 | 2.3 | 0 | 0 | 3.0 | 3.0 |
| min E | | | | | | | | | |
| Flavor- | | | | | 1.0 | 1.0 | 2.0 | 1.0 | 1.0 |
| ing | | | | | | | | | |

¹EPA—eicosapentaenoic acid
²DHA—docosahexaenoic acid
³OLA—oleic acid
⁴LNA—linoleic acid
⁵CLA—conjugated linoleic acid

### Example 30-39

Exemplary compositions including gel capsules may be prepared as described above including the ingredients listed in Table 4.

| | Ex. | Ex. | Ex. | Ex. | Ex. | Ex. | Ex. | Ex. | Ex. | Ex. |
|---|---|---|---|---|---|---|---|---|---|---|
| Compound | 30 | 31 | 32 | 33 | 34 | 35 | 36 | 37 | 38 | 39 |
| EPA¹ | 200 | 200 | 200 | 200 | 200 | 200 | 200 | 200 | 200 | 200 |
| DHA² | 200 | 200 | 200 | 200 | 200 | 200 | 200 | 200 | 200 | 200 |
| NO-OLA³ | 100 | 100 | 100 | 100 | 100 | 100 | 100 | 100 | 100 | 100 |
| NO-LNA⁴ | 100 | 100 | 100 | 100 | 100 | 100 | 100 | 100 | 100 | 100 |
| NO-CLA⁵ | 100 | 100 | 100 | 100 | 100 | 100 | 100 | 100 | 100 | 100 |
| Vitamin E | 3 | 3 | 3 | 3 | 0 | 0 | 0 | 0 | 0 | 0 |
| Vitamin B12 | 20 | 20 | 0 | 0 | 20 | 20 | 0 | 0 | 0 | 20 |
| Folic Acid | 0 | 0.8 | 0 | 0 | 0 | 0.8 | 0 | 0 | 0 | 0.8 |
| Ginko Biloba | 0 | 0 | 400 | 0 | 0 | 0 | 400 | 0 | 400 | 400 |
| Ginseng | 0 | 0 | 0 | 200 | 0 | 0 | 0 | 200 | 200 | 200 |
| Flavoring | 2.0 | 2.0 | 2.0 | 2.0 | 2.0 | 2.0 | 2.0 | 2.0 | 2.0 | 2.0 |

¹EPA—eicosapentaenoic acid
²DHA—docosahexaenoic acid
³OLA—oleic acid
⁴LNA—linoleic acid
⁵CLA—conjugated linoleic acid

### Example 40

Topical compositions may be prepared including:

0.01% w/w LNO₂, 0.01% w/w OA NO₂, 2.00 w/w % PEG-400, 2.00% w/w Labrasol, 1.00% w/w Oleth-3, 1.00% w/w Diglycerin, 0.01% w/w Oxynex AP and PEG-200 q.s. to 100% w/w.

0.025% w/w LNO₂, 0.01% w/w OA NO₂, 5.00 w/w % PEG-400, 2.00% w/w Propylene glycol, 1.00% w/w BV-OSC, 1.00% w/w VC-IP, 0.25% w/w Beta carotene, 5.00% w/w Diisopropyl adipate 0.01% w/w Oxynex AP and PEG-200 q.s. to 100% w/w.

0.02% w/w LNO₂, 0.10% w/w Phytantriol, 0.1% w/w Panthenol, 10.00% w/w, 10.00% w/w Glycereth 4, 5.00% w/w Ceraphyl® 41, 1.00% w/w Oleth 3 0.01% w/w Oxynex AP and Diglycerin q.s. to 100% w/w.

0.01% w/w CLA-NO₂, 0.01% w/w OA NO₂, 2.00 w/w % PEG-400, 2.00% w/w Labrasol, 1.00% w/w Oleth-3, 1.00% w/w Diglycerin, 0.01% w/w Oxynex AP and PEG-200 q.s. to 100% w/w.

0.025% w/w CLA-NO₂, 0.01% w/w OA NO₂, 5.00 w/w % PEG-400, 2.00% w/w Propylene glycol, 1.00% w/w BV-OSC, 1.00% w/w VC-IP, 0.25% w/w Beta carotene, 5.00% w/w Diisopropyl adipate 0.01% w/w Oxynex AP and PEG-200 q.s. to 100% w/w.

0.02% w/w CLA-NO₂, 0.10% w/w Phytantriol, 0.1% w/w Panthenol, 10.00% w/w, 10.00% w/w Glycereth 4, 5.00% w/w Ceraphyl® 41, 1.00% w/w Oleth 3 0.01% w/w Oxynex AP and Diglycerin q.s. to 100% w/w.

Topical emulsion compositions may be prepared including:

0.01% w/w LNO₂, 15.00% w/w Ethoxylated glycerin, 5.00% w/w Glycerin, 0.10% w/w NaCl, 1.00% w/w BV-OSC, 35.00% Mineral oil, 2.00% w/w Dow Corning® Fluid 244 (methylsiloxane fluid), 5.00% w/w Abil WE-09 (Polygrycerol-4 isostearate and cetyl dimethicone cpolyol and hexyl laureate), 1.00% w/w Cranberry seed oil, 0.01% w/w Oxynex AP and PEG-400 q.s. to 100% w/w.

0.01% w/w OA NO₂, 15.00% w/w Ethoxylated glycerin, 5.00% w/w Glycerin, 0.10% w/w NaCl, 1.00% w/w BV-OSC, 35.00% Mineral oil, 2.00% w/w Dow Corning® Fluid 244 (methylsiloxane fluid), 5.00% w/w Abil WE-09 (Polygrycerol-4 isostearate and cetyl dimethicone cpolyol and hexyl laureate), 1.00% w/w Cranberry seed oil, 0.01% w/w Oxynex AP and PEG-400 q.s. to 100% w/w.

0.025% w/w LNO₂, 5.00% w/w Glycerin, 0.0% w/w NaCl, 1.00% w/w BV-OSC, 35.00% Mineral oil, 2.00% w/w Dow Corning® Fluid 244 (methylsiloxane fluid), 1.00% w/w Cranberry seed oil, 0.01% w/w Oxynex AP and PEG-400 q.s. to 100% w/w.

0.025% w/w OA NO₂, 5.00% w/w Glycerin, 0.10% w/w NaCl, 1.00% w/w BV-OSC, 35.00% Mineral oil, 2.00% w/w Dow Corning® Fluid 244 (methylsiloxane fluid), 1.00% w/w Cranberry seed oil, 0.01% w/w Oxynex AP and PEG-400 q.s. to 100% w/w.

0.01% w/w LNO₂, 5.00% w/w Glycerin, 0.10% w/w NaCl, 15.00% w/w Dow Corning® Fluid 245 (cyclopentasilxane fluid), 9.00% w/w Dow Corning® Fluid 3225 C (silicone surfactant in dimethylsiloxane), 1.50% w/w Tween 2, 0.01% w/w Oxynex AP and PEG-400 q.s. to 100% w/w.

0.01% w/w OA NO₂, 5.00% w/w Glycerin, 0.10% w/w NaCl, 15.00% w/w Dow Corning® Fluid 245 (cyclopentasilxane fluid), 9.00% w/w Dow Corning® Fluid 3225 C (silicone surfactant in dimethylsiloxane), 1.50% w/w Tween 2, 0.01% w/w Oxynex AP and PEG-400 q.s. to 100% w/w.

0.01% w/w CLA-NO₂, 15.00% w/w Ethoxylated glycerin, 5.00% w/w Glycerin, 0.10% w/w NaCl, 1.00% w/w BV-OSC, 35.00% Mineral oil, 2.00% w/w Dow Corning® Fluid 244 (methylsiloxane fluid), 5.00% w/w Abil WE-09 (Polygrycerol-4 isostearate and cetyl dimethicone cpolyol and hexyl laureate), 1.00% w/w Cranberry seed oil, 0.01% w/w Oxynex AP and PEG-400 q.s. to 100% w/w.

0.025% w/w CLA-NO₂, 5.00% w/w Glycerin, 0.10% w/w NaCl, 1.00% w/w BV-OSC, 35.00% Mineral oil, 2.00% w/w Dow Corning® Fluid 244 (methylsiloxane fluid), 1.00% w/w Cranberry seed oil, 0.01% w/w Oxynex AP and PEG-400 q.s. to 100% w/w.

Topical compositions and emulsions can be applied to areas of the skin such as the face at established intervals resulting in a gradual improvement in the skin areas with each successive application.

Topically applied compositions are absorbed by the skin and can inhibit inflammation. Therefore, topical compositions of the present invention are expected to be particularly useful in the prevention and treatment of conditions including: rosacea, eczema, psoriasis, xerosis, dermatitis (contact and atopic), sebhorrea, thermal and radiation burns (including sunburn), acne, alopecia, aging-induced skin tissue degeneration, scars, and other conditions associated with skin inflammation.

It is expected that the compositions of the present invention will also be useful in the treatment and prevention of alopecia, where skin inflammation is frequently present.

### Example 41

A gel capsule nutraceutical may be prepared including: 150 mg Borage oil, 30 mg nitrated gamma linolenic acid, 1,000 mg fish body oil, 180 mg nitrated DHA, 120 mg EPA, 5 mg rosemary extract, 20 mg lemon flavor, 5 IU vitamin E, and 5 mcg Coenzyme Q-10.

### Example 42

A gel capsule nutraceutical may be prepared including: 150 mg Borage oil, 30 mg nitrated gamma linolenic acid, 75 mg oleic acid, 75 mg olive oil, 25 mg liquid soy lecithin, 133 mg phytosterol ester, 400 mg fish body oil, 72 mg nitrated EPA, 48 mg nitrated DHA, 12 mg DHA, 33 IU vitamin E, 0.5 mg palm oil, 0.5 mg raspberry oil, 0.5 mg cranberry oil, 8.5 mg rice bran oil, 1.7 mg tocotrienols, 20 mg Coenzyme Q-10, and 10 mg natural lemon flavor.

### Example 43

A gel capsule nutraceutical may be prepared including: 150 mg Borage oil, 30 mg nitrated gamma linolenic acid, 75 mg oleic acid, 75 mg olive oil, 25 mg liquid soy lecithin, 133 mg phytosterol ester, 400 mg fish body oil, 72 mg nitrated EPA, 48 mg nitrated DHA, 12 mg DHA, 33 IU vitamin E, 400 mg Ginko Bilbo, 8.5 mg green tea extract, 0.5 mg palm oil, 0.5 mg raspberry oil, 0.5 mg cranberry oil, 1.7 mg tocotrienols, 20 mg Coenzyme Q-10, and 10 mg natural lemon flavor.

### Example 44

A gel capsule nutraceutical may be prepared including: 150 mg Borage oil, 30 mg nitrated gamma linolenic acid, 75 mg oleic acid, 75 mg olive oil, 25 mg liquid soy lecithin, 133 mg phytosterol ester, 400 mg fish body oil, 72 mg nitrated EPA, 48 mg nitrated DHA, 12 mg DHA, 33 IU vitamin E, 400 mg Ginko Bilbo, 20 mg Vitamin B12, 0.8 mg Folic Acid, 0.5 mg palm oil, 0.5 mg raspberry oil, 0.5 mg cranberry oil, 8.5 mg rice bran oil, 1.7 mg tocotrienols, 20 mg Coenzyme Q-10, and 10 mg natural lemon flavor.

### Example 45

A gel capsule nutraceutical may be prepared including: 150 mg Borage oil, 30 mg conjugated nitrated linoleic acid, 75 mg oleic acid, 75 mg olive oil, 25 mg liquid soy lecithin, 133 mg phytosterol ester, 400 mg fish body oil, 72 mg nitrated EPA, 48 mg nitrated DHA, 12 mg DHA, 33 IU vitamin E, 400 mg Ginko Bilbo, 20 mg Vitamin B12, 0.8 mg Folic Acid, 0.5 mg palm oil, 0.5 mg raspberry oil, 0.5 mg cranberry oil, 8.5 mg rice bran oil, 1.7 mg tocotrienols, 20 mg Coenzyme Q-10, and 10 mg natural lemon flavor.

### Example 46

Stability experiments were conducted using Bertolli extra light olive oil. Methanol soluble impurities were extracted twice. 750 uM OA-NO₂ dissolved in methanol was added to the olive oil and incubated for 19 days at 22° C., 37° C. and 50° C. Olive oil is highly hydrophobic and liposomes have been previously shown to stabilize nitrated linoleic acid. The composition of Olive oil used was as follows: total fat: 14 g, saturated fat: 9%, polyunsaturated fat: 9%, Monounsaturated Fat: 45%

First, methanol-soluble substances were extracted from olive oil by applying 6 volumes of methanol per volume of oil and vortexing for a minute. The triglyceride containing fraction was obtained and the extraction was repeated. The remaining triglycerides were dried under vacuum to eliminate remaining organic solvents. Once dry, a concentrated solution of nitrated oleic acid (dissolved in a small volume of ethanol) was added to reach a final concentration of 750 M. Method of detection.

Samples were then stored in the dark at 22° C., 37° C. and 50° C. A bracketing study for humidity was performed in which the samples at 22° C. and 37° C. were subjected to humidity conditions normally found in the Midwestern United States while the samples at 50° C. were subjected to water-saturated air in an enclosed water bath at 50° C.

At the different time points, aliquot by triplicate were obtained, fatty acids were extracted from the triglyceride matrix using a 4:1 ratio of methanol:triglyceride, diluted to a final concentration of 100 nM and quantified using liquid chromatography coupled to mass spectrometry. The sample (10 μl) was injected using an automated Shimadzu autosampler and HPLC pumps (SIL20 System), and chromatographically separated using a C18 reverse phase column (2 mm Mercury cartridge columns, Phenomenex). The nitrated oleic acid was detected using the multiple reaction monitoring (MRM) process in the negative ion mode performed on a 4000 QTRAP triple quadrupole (Applied Biosystems). The selected MRM corresponded to the formation of the product ion nitrite from the nitrated oleic acid, having a specific transition of 324.3/46. The HPLC method was based on solvent A (H2O with 0.1% Acetic Acid) and B (Acetonitrile with 0.1% Acetic Acid). A gradient was developed starting at 35% B over 6 min to reach 100% B. The column was then washed at 100% B for 2 minutes and re-equilibrated at initial conditions for 3 minutes. The flow rate was established at 750 μl/min. The peak areas were integrated using Analyst 1.5.1 software (Applied Biosystems) and external standard curves were performed for quantification purposes.

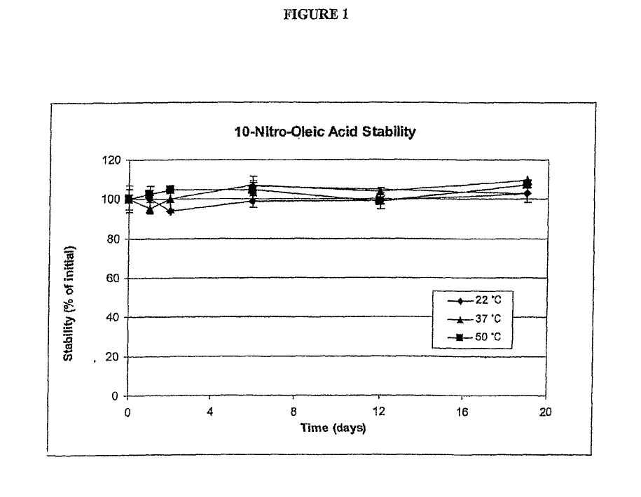

FIG. 1 shows stability of 10-nitro oleic acid in olive oil over a period of 19 days at 22° C., 37° C. and 50° C. Stability is plotted as a percentage of the starting concentration of 10-nitro oleic acid. 10-nitro oleic acid is shown to be stable in olive at a range of temperatures for periods of up to 20 days. After 135 days, stability was found to be decreased by about 15% in samples incubated at 22° C., 37° C.

### Example 47

A number of alimentary fats including plant oils such, olive oil (virgin, and refined), sunflower oil, vegetable oil flax seed oil, sesame oil, palm oil, soybean oil, canola oil, pumpkin seed oil, corn oil, safflower oil, peanut oil, grape seed oil, argan oil, avocado oil, mustard oil, Almond oil, cottonseed oil, diacylglycerol (DAG) oil, ghee, Walnut oil, rice bran oil as well as other vegetable oils contain abundant amount of unsaturated fatty acids and are suitable for human consumption. Unsaturated fatty acids can also be found in relative abundance in animal fats such as clarified butter, lard and fish oils such as cod liver oil and herring oil.

These unsaturated fatty acids are expected to be readily converted to nitro fatty acid include by contacting existing unsaturated fatty acid with a nitro containing compound; and reacting an existing unsaturated fatty acid with a nitro containing compound to form a nitro fatty acid. Foodstuffs enriched for nitro fatty acids are expected to improve the health of an individual as part of a balanced diet.

Activated fatty acids may be prepared by a method including the steps of reacting the unsaturated fatty acid with a mercuric salt (such as, for example, HgCl₂, Hg(NO₃)₂, Hg(OAc)₂) and a selenium compound (including but not limited to PhSeBr, PhSeCl, PhSeO₂CCF₃, PhSeO₂H, PhSeCN), contacting the intermediate resulting from step a) with a reagent or reactant that can introduce an electron withdrawing group; and reacting the intermediate resulting from step b) with an oxidizing agent (including but not limited to oxygen (O2), ozone (O3), hydrogen peroxide (H2O2) and other inorganic peroxides, Fluorine (F2), chlorine (Cl2), and other halogens, nitric acid (HNO3) and nitrate compounds, sulfuric acid (H2SO4), persulfuric acids (H2SO5 and H2SO8), chlorite, chlorate, perchlorate, and other analogous halogen compounds, hypochlorite and other hypohalite compounds, including bleach (NaClO), hexavalent chromium compounds such as chromic and dichromic acids and chromium trioxide, pyridinium chlorochromate (PCC), and chromate/dichromate compounds, prmanganate compounds sodium perborate, nitrous oxide (N2O) silver oxide (Ag2O), osmium tetroxide (OsO4), Tollens' reagent, 2,2′-dipyridyldisulfide (DPS) sodium periodate, meta-Chloroperoxybenzoic acid and t-butyl hydroperoxide.

The source of the electron withdrawing group may be any compound known in the art that is capable of generating an electron withdrawing group that can be incorporated into the activated fatty acid, such as, for example, NaNO₂, AgNO₂, HSO₂OH. In certain embodiments, the process of forming nitrated fatty acids is performed in the absence of oxygen.

### Example 48

It is envisioned that a variety of alimentary fats as vegetable oils and animal fat can be treated with nitrite to produce activated fatty acids from polyunsaturated fatty acids such as linoleic acid, conjugated-linoleic acid, α-linoleic acid, γ-linoleic acid, oleic acid, eicosapentaenoic acid (EPA), docosahexaenoic acid (DHA), or derivatives thereof.

80% of the basal plasma nitrite (NO₂) level derives from oxidation of NO, reduction of peroxynitrate (NO₃⁻) may also contribute to elevation of NO₂⁻. It has been reported that exogenous NO₃⁻ intake (10 mg/kg in humans) may increase plasma NO₂⁻ concentration up to four-to five-fold in 30 min.

The largest dietary sources of NO₃ for the human body include green vegetables, such as spinach, lettuce, and collard greens, and also radishes, beets and, and meat. Furthermore, NO₂⁻ itself can be found in cured meats.

Nitrated lipids or activated fatty acids can be formed by several different mechanisms, such as through a reaction of NO₂ with unsaturated fatty acid derivatives at low pH. For example, The “Mediterranean diet”, which is particularly rich in NO₂⁻ and PUFA, and supplemented with acidic vinegar, may favor intragastric generation of nitrated lipids. Indeed, it has been shown that nitration of unsaturated fatty acids from extra virgin olive oil is possible under exposure to NO₂⁻ in mild acidic conditions.

Recent research by d'Ischia and colleagues has demonstrated that oxides of nitric oxide and its oxidation products including NO₃⁻, NO₂, HNO₂ and NO₂⁺, interact with unsaturated fatty acid such as linoleic acid and lipid peroxides to produce complex mixtures of products, including nitroepoxides and other nitrogen containing derivatives of oxidized lipids. At high concentrations and in the presence of oxygen, NO reacts with polyunsaturated fatty acids and esters to afford mixtures of nitration products, including isomeric nitroalkene and nitronitrate derivatives. For example, ethyl linoleate reacts smoothly with NO₂⁻ in acidic media, conditions that favor formation of HNO₂, to afford complex, yet relatively well-defined patterns of nitration products, some of which were amenable to chromatographic isolation.

The presence of oxygen can have a dramatic effect on the nitration of unsaturated fatty acids. Species such as nitric oxide are highly lipophilic and will more readily react molecular oxygen that also preferentially partions into a hydrophobic milieu. In some embodiments, it is desirable to conduct the nitration of unsaturated fatty acids in the absence of molecular oxygen, such that the latter does not compete for free radicals with the fatty acid substrates. It is further expected that some of the initial unsaturated fatty acids will not become nitrated and will remain in their native state.

In a particular embodiment, Olive oil, which contains abundant amounts of unsaturated fatty acids (85% oleic acid and 5% linoleic acid) represents suitable substrate for nitration with NO₂⁻. It is expected that the nitration of olive will yield a complex mixture of fatty acids including nitro-oleic acid, nitro-linoleic acid, oleic acid and linoleic acid. Based on the relative ratio of oleic acid to linoleic acid of about 18:1 it is expected that the ratio of nitrated oleic acid to linoleic acid will be similar. The higher the free fatty acid content of an oil the greater the acidity and therefore the more suitable such oils are to being nitrated. It is further expected that some of the initial unsaturated fatty acids will not become nitrated and will remain in their native state.

In another embodiment, fish oil is a suitable substrate for nitration. The typical composition of unsaturated fatty acids in fish oil is docosahexaenoic acid, eicosapentaenoic acid, linoleic acid, oleic acid. The ratios of oleic acid to linoleic acid to docosahexaenoic acid and eicosapentaenoic acid combined are about 1:10:26. The ratio of docosahexaenoic acid to eicosapentaenoic acid in turn is typically about 5:1.

In yet other embodiments, corn oil, palm oil, peanut oil and safflower oil also make suitable substrates for nitration due to high levels of oleic and, linoleic acid and conjugated linoleic acid.

In addition to fish oil and olive oil, safflower oil comprises upwards of 80% conjugated linoleic acid and also contains about 20%. It is therefore expected that nitration of sallfower oil will result in the formation of conjugated nitro-linoleic acid and nitro-oleic acid at a ratio of about 4:1 under anaerobic conditions. It is further expected that some of the initial polyunsaturated fatty acids will not become nitrated and will remain in their native state.

It is envisioned that a alimentary oil such as olive oil, that is already known to impart overall health benefits to people that consume it regularly as part of their diet, will provide additional overall health benefits due to the presence of nitrated fatty acids as many of the health benefits observed may result from the nitration of unsaturated fatty acids in the gut upon consumption.

### Example 49

A 31 year old male with type-2 diabetes was given 2600 mg per day of conjugated linoleic acid for a two month period along with patients routine therapy to control his blood sugar consisting of a liraglutide and repaglinide. The subject reported about 20% lower fasting blood glucose when taking the conjugated linoleic acid. In addition, the subject reported that he lost approximately 8-10 pounds during the 60 day trial. The subject exercised regularly and adhered to a normal diet during the course of the trial. The results from this single subject trial suggest that conjugated linoleic acid may promote weight loss and aid in lowering fasting blood glucose levels.

1.-101. (canceled)

102. A composition comprising an effective amount of at least one activated fatty acid and an alimentary oil.

103. The composition of claim 102, wherein an alimentary oil is either a plant oil, an animal fat or a combination thereof.

104. The composition of claim 102 wherein the activated fatty acid is nitrated α-linoleic acid, nitrated γ-linoleic acid, nitrated oleic acid, nitrated eicosapentaenoic acid, nitrated docosahexaenoic acid or a derivative or combination thereof.

105. The composition of claim 102, wherein the alimentary oil is olive oil.

106. A method of nitrating an alimentary oil comprising contacting the alimentary oil with an oxide of nitrogen.

107. The method of claim 106 wherein, the alimentary oil is olive oil, flaxseed oil, fish oil or a combination thereof.

108. The method of claim 106 wherein the oxide of nitrogen is NO, NO3−, NO2, HNO2, NO2+ or combination thereof.

109. The method of claim 106, wherein nitration occurs at an acidic pH.

110. The method of claim 109, wherein the pH is less than 4.0.

111. A composition comprising: an alimentary oil, one or more nitrated fatty acid and one or more unsaturated fatty acid.

112. The composition of claim 111 wherein the alimentary oil is olive oil, the nitrated fatty acids are nitro-linoleic acid, nitro-oleic acid or a combination thereof and the unsaturated fatty acids are linoleic acid, oleic acid or a combination thereof.

113. (canceled)

114. (canceled)

115. The composition of claim 111, wherein the alimentary oil is flaxseed oil, the nitrated fatty acids are nitro-linoleic acid, nitro-oleic acid or a combination thereof and the unsaturated fatty acids are linoleic acid, oleic acid or a combination thereof.

116. (canceled)

117. (canceled)

118. The composition of claim 111, wherein the alimentary oil is docosahexaenoic acid, the nitrated fatty acid is nitro-docosahexaenoic acid and the unsaturated fatty acid is docosahexaenoic acid.

119. The composition of claim 111, wherein the alimentary oil is fish oil, the nitrated fatty acid is nitro-docosahexaenoic acid, nitrated eicosapentaenoic acid, nitro-linoleic acid, nitro-oleic acid or a combination thereof and the unsaturated fatty acid is docosahexaenoic acid, eicosapentaenoic acid, linoleic acid, oleic acid or a combination thereof.

120.-125. (canceled)

126. The composition of claim 111, wherein the alimentary oil is safflower oil, the nitrated fatty acid is conjugated linoleic acid, nitro-linoleic acid, nitro-oleic acid or a combination thereof and the unsaturated fatty acid is conjugated linoleic acid, linoleic acid, oleic acid or a combination thereof.

127.-151. (canceled)
